(19) United States
(12) Patent Application Publication
(10) Pub. No.: US 2013/0065222 A1
(43) Pub. Date: Mar. 14, 2013

(54) COMPOSITIONS, METHODS AND REACTION MIXTURES FOR THE DETECTION OF MURINE LEUKEMIA VIRUS-RELATED VIRUS

(71) Applicants: Kui GAO, San Diego, CA (US); Jeffrey M. LINNEN, Poway, CA (US); Kurt Craft NORTON, La Mesa, CA (US)

(72) Inventors: Kui GAO, San Diego, CA (US); Jeffrey M. LINNEN, Poway, CA (US); Kurt Craft NORTON, La Mesa, CA (US)

(73) Assignee: GEN-PROBE INCORPORATED, San Diego, CA (US)

(21) Appl. No.: 13/598,182

(22) Filed: Aug. 29, 2012

Related U.S. Application Data
(60) Provisional application No. 61/529,189, filed on Aug. 30, 2011.

(51) Int. Cl. C12Q 1/70

(52) U.S. Cl. 435/5

(57) ABSTRACT

The present invention relates to the detection of infectious agents, more specifically to the detection of murine leukemia viruses and other highly related viruses, including but not limited to ecotropic murine leukemia viruses, xenotropic murine leukemia viruses, and polytropic murine leukemia viruses. Compositions, methods, reaction mixtures and kits are described for the detection of MLV by using in vitro nucleic acid amplification techniques.

### CROSS REFERENCE TO RELATED APPLICATIONS

This application claims the benefit of and priority to U.S. provisional application 61/529,189, filed Aug. 30, 2011, the entirety of which is hereby incorporated by reference.

### FIELD OF THE INVENTION

The present invention relates to the detection of infectious agents, more specifically to the detection of murine leukemia viruses and other highly related viruses, including but not limited to ecotropic murine leukemia viruses, xenotropic murine leukemia viruses, and polytropic murine leukemia viruses. Compositions, methods, reaction mixtures and kits are described for the detection of MLV by using in vitro nucleic acid amplification techniques.

### BACKGROUND

Murine leukemia viruses (MLV) are retroviruses that are capable of causing cancer in mice. MLV can be transmitted from one host to another (exogenous) or from one generation to another (endogenous). Endogenous MLV are further classified by host specificity, that is MLV that are capable of only infecting mice cells (ecotropic), MLV that are capable of only infecting non-mice cells (xenotropic) and MLV that are capable of infecting mice and non-mice cells (polytropic).

### SUMMARY OF THE INVENTION

The present invention relates to the detection of infectious agents, more specifically to the detection of murine leukemia viruses and other highly related viruses, including but not limited to ecotropic murine leukemia viruses, xenotropic murine leukemia viruses, and polytropic murine leukemia viruses, all herein referred to as “MLV”. Compositions, methods, reaction mixtures, and kits are described for the detection of MLV by using in vitro nucleic acid amplification techniques.

One embodiment provides a method for the amplification and identification of an MLV from a sample comprising the steps of: contacting a sample suspected of containing MLV with at least two amplification oligomers for generating an amplicon, wherein each of said at least two amplification oligomers is from about 10 to about 50 nucleobases in length and wherein said at least two amplification oligomers are respectively configured to specifically hybridize to regions within a target sequence of MLV selected from the group consisting of from residue 2800 to residue 2862 and from residue 2924 to residue 2971 of SEQ ID NO:85, or from residue 7676 to residue 7713 and from residue 7756 to residue 7804 of SEQ ID NO:85; providing conditions sufficient for generating an amplicon from an MLV target nucleic acid present in said sample using said amplification oligomers; and providing conditions for detecting said amplicon and determining whether said sample contains MLV target nucleic acid.

In one aspect, at least one of said at least two amplification oligomers comprises a target binding sequence that contains a sequence selected from the group consisting of SEQ ID NOS: 99, 102, 103, 109, 125, 127, 130, 131, 157, 158, and 159. In another aspect, the at least two amplification oligomers comprise a first amplification oligomer comprising, consisting or consisting essentially of a target binding sequence containing SEQ ID NOS: 99, 102, or 103, and a second amplification oligomer comprising, consisting, or consisting essentially of a target binding sequence containing SEQ ID NOS: 125, 127, or 157. In another aspect, the at least two amplification oligomers comprise a first amplification oligomer comprising, consisting or consisting essentially of a target binding sequence containing SEQ ID NOS: 109 or 158, and a second amplification oligomer comprising, consisting, or consisting essentially of a target binding sequence containing SEQ ID NOS: 130, 131, or 159. In another aspect, the at least two amplification oligomers comprise a first amplification oligomer comprising, consisting or consisting essentially of a target binding sequence as set forth in any one of SEQ ID NOS: 97 to 104 and a second amplification oligomer comprising, consisting or consisting essentially of a target binding sequence as set forth in any one of SEQ ID NOS: 122 to 127. In another aspect, the at least two amplification oligomers comprise a first amplification oligomer comprising, consisting or consisting essentially of a target binding sequence as set forth in any one of SEQ ID NOS: 105 to 109 and a second amplification oligomer comprising, consisting or consisting essentially of a target binding sequence as set forth in any one of SEQ ID NOS: 128 to 133. In another aspect, the at least two amplification oligomers are one of SEQ ID NOS: 97 to 104; and one of SEQ ID NOS: 110 to 115 or 122 to 127. In another aspect, the at least two amplification oligomers are one of SEQ ID NOS: 105 to 109; and one of SEQ ID NOS: 116 to 121 or 128 to 133.

In one aspect, the amplicon is detected using a detection probe oligomer comprising a target binding sequence set forth in any one of SEQ ID NOS: 148 to 150. In another aspect, the amplicon is detected using a detection probe oligomer comprising a target binding sequence set forth in any one of SEQ ID NOS: 151 to 156

One embodiment provides a method for the multiplex amplification and identification of an MLV from a sample comprising the steps of: contacting a sample suspected of containing MLV with at least two amplification oligomer pairs for generating separate amplicons from an MLV target nucleic acid, wherein each amplification oligomer of said at least two amplification oligomer pairs is from 10 to about 50 nucleobases in length and wherein a first amplification oligomer pair is configured to specifically hybridize to regions within a target sequence of MLV comprising, consisting or consisting essentially of from residue 2800 to residue 2862 and from residue 2924 to residue 2971 of SEQ ID NO:85, and wherein a second amplification oligomer pair is configured to specifically hybridize to regions within a target sequence of MLV comprising, consisting or consisting essentially of from residue 7676 to residue 7713 and from residue 7756 to residue 7804 of SEQ ID NO:85; providing conditions sufficient for generating amplicons from an MLV target nucleic acid present in said sample using said amplification oligomers; and providing conditions for detecting said amplicon and determining whether said sample contained MLV target nucleic acid.

In one aspect of this multiplex reaction, at least one amplification oligomer of said at least two amplification oligomer pairs comprises a target binding sequence that contains a sequence selected from the group consisting of SEQ ID NOS: 99, 102, 103, 109, 125, 127, 130, 131, 157, 158, and 159. In another aspect, the amplification oligomer pair comprises a first amplification oligomer comprising, consisting or consisting essentially of a target binding sequence containing SEQ ID NOS: 99, 102, or 103 and a second amplification oligomer comprising, consisting or consisting essentially of a target binding sequence containing SEQ ID NOS: 125, 127, or 157. In another aspect, the amplification oligomer pair comprises a first amplification oligomer comprising, consisting or consisting essentially of a target binding sequence containing SEQ ID NOS: 109 or 158 and a second amplification oligomer comprising, consisting or consisting essentially of a target binding sequence containing SEQ ID NOS: 130, 131, or 159. In another aspect, the amplification oligomer pair comprises a first amplification oligomer comprising, consisting or consisting essentially of a target binding sequence as set forth in any one of SEQ ID NOS: 97 to 104 and a second amplification oligomer comprising, consisting or consisting essentially of a target binding sequence as set forth in any one of SEQ ID NOS:122 to 127. In another aspect, the amplification oligomer pair comprises a first amplification oligomer comprising, consisting or consisting essentially of a target binding sequence as set forth in any one of SEQ ID NOS: 105 to 109 and a second amplification oligomer comprising, consisting or consisting essentially of a target binding sequence as set forth in any one of SEQ ID NOS: 128 to 133. In another aspect, the first amplification oligomer pair is one of SEQ ID NOS: 97 to 104 and one of SEQ ID NOS: 110 to 115 or 122 to 127. In another aspect, the second amplification oligomer pair is one of SEQ ID NOS: 105 to 109 and one of SEQ ID NOS: 116 to 121 or 128 to 133.

In one aspect of this multiplex reaction, the amplicon is detected using a detection probe oligomer comprising a target binding sequence set forth in SEQ ID NO: 148 to 150. In another aspect, the amplicon is detected using a detection probe oligomer comprising a target binding sequence set forth in SEQ ID NO: 151 to 156.

In one aspect, the amplification reaction is substantially isothermal. In one aspect, the amplification reaction is PCR. In one aspect, the amplification reaction is transcription based. In one aspect, the amplification reaction is TMA. In one aspect, the amplicon is detected in real-time. In one aspect, the amplicon is detected at the end of the amplification reaction. In one aspect, the amplicon is detected using a method such as sequencing, mass spectrometry, detection probe based detection, or other known technique. Detection probe based detection includes, but is not limited to, chemiluminescent labelled detection probe oligomers, or fluorophore:quencher labelled detection probe oligomers. In one aspect, the amplicon is detected using a detection probe oligomer. In one aspect, the detection probe oligomer is labelled with a chemiluminescent compound. In one aspect, the detection probe oligomer is labelled with an AE compound.

In one aspect, the sample is human blood donated for transfusion into an individual. In another aspect, the sample is human blood donated for use by a human blood bank. In one aspect, the sample is human blood.

One embodiment provides a composition or a reaction mixture for use in an MLV target nucleic acid amplification assay comprising at least two amplification oligomers capable of stably hybridizing to MLV target nucleic acid, wherein each amplification oligomer of said at least two amplification oligomers is from about 10 to about 50 nucleobases in length, and wherein said at least two amplification oligomers are respectively configured to specifically hybridize to regions within a target sequence of MLV selected from the group consisting of from residue 2800 to residue 2862 and from residue 2924 to residue 2971 of SEQ ID NO: 85; and from residue 7676 to residue 7713 and from residue 7756 to residue 7804 of SEQ ID NO: 85.

In one aspect of the composition or reaction mixture, at least one amplification oligomer of said at least two amplification oligomers comprises a target binding sequence that contains a sequence selected from the group consisting of SEQ ID NOS: 99, 102, 103, 109, 125, 127, 130, 131, 157, 158, and 159. In another aspect, the at least two amplification oligomers comprise a first amplification oligomer comprising, consisting or consisting essentially of a target binding sequence containing SEQ ID NOS: 99, 102, or 103, and a second amplification oligomer comprising, consisting, or consisting essentially of a target binding sequence containing SEQ ID NOS: 125, 127, or 157. In another aspect, the at least two amplification oligomers comprise a first amplification oligomer comprising, consisting or consisting essentially of a target binding sequence containing SEQ ID NOS: 109 or 158, and a second amplification oligomer comprising, consisting, or consisting essentially of a target binding sequence containing SEQ ID NOS: 130, 131, or 159. In another aspect, the at least two amplification oligomers comprise a first amplification oligomer comprising, consisting or consisting essentially of a target binding sequence as set forth in any one of SEQ ID NOS: 97 to 104 and a second amplification oligomer comprising, consisting or consisting essentially of a target binding sequence as set forth in any one of SEQ ID NOS: 122 to 127. In another aspect, the at least two amplification oligomers comprise a first amplification oligomer comprising, consisting or consisting essentially of a target binding sequence as set forth in any one of SEQ ID NOS: 105 to 109 and a second amplification oligomer comprising, consisting or consisting essentially of a target binding sequence as set forth in any one of SEQ ID NOS: 128 to 133. In another aspect, the at least two amplification oligomers are one of SEQ ID NOS: 97 to 104; and one of SEQ ID NOS: 110 to 115 or 122 to 127. In another aspect, the at least two amplification oligomers are one of SEQ ID NOS: 105 to 109; and one of SEQ ID NOS: 116 to 121 or 128 to 133.

In one aspect, the composition or reaction mixture further includes a detection probe oligomer wherein said detection probe oligomer comprises a target binding sequence set forth in any one of SEQ ID NOS: 148 to 150. In another aspect, the composition or reaction mixture further includes a detection probe oligomer wherein said detection probe oligomer comprises a target binding sequence set forth in any one of SEQ ID NO: 151 to 156.

One embodiment provides a composition or a reaction mixture for use in an MLV target nucleic acid multiplex amplification assay comprising at least two amplification oligomer pairs capable of stably hybridizing to an MLV target nucleic acid, wherein each amplification oligomer of a first amplification oligomer pair is from about 10 to about 50 nucleobases in length and is configured to specifically hybridize to regions within a target sequence of MLV comprising, consisting or consisting essentially of from residue 2800 to residue 2862 and from residue 2924 to residue 2971 of SEQ ID NO: 85, and wherein each amplification oligomer of a second amplification oligomer pair is from about 10 to about 50 nucleobases in length and is configured to specifically hybridize to regions within a target sequence of MLV comprising, consisting or consisting essentially of from residue 7676 to residue 7713 and from residue 7756 to residue 7804 of SEQ ID NO: 85.

In one aspect of the composition or reaction mixture, at least one amplification oligomer of said at least two amplification oligomer pairs comprises a target binding sequence that contains a sequence selected from the group consisting of SEQ ID NOS: 99, 102, 103, 109, 125, 127, 130, 131, 157, 158, and 159. In another aspect, the first amplification oligomer pair comprises a first amplification oligomer comprising, consisting or consisting essentially of a target binding sequence containing SEQ ID NOS: 99, 102, or 103 and a second amplification oligomer comprising, consisting or consisting essentially of a target binding sequence containing SEQ ID NOS: 125, 127, or 157. In another aspect, the second amplification oligomer pair comprises a first amplification oligomer comprising, consisting or consisting essentially of a target binding sequence containing SEQ ID NOS: 109 or 158 and a second amplification oligomer comprising, consisting or consisting essentially of a target binding sequence containing SEQ ID NOS: 130, 131, or 159. In another aspect, the first amplification oligomer pair comprises a first amplification oligomer comprising, consisting or consisting essentially of a target binding sequence as set forth in any one of SEQ ID NOS: 97 to 104 and a second amplification oligomer comprising, consisting or consisting essentially of a target binding sequence as set forth in any one of SEQ ID NOS: 122 to 127. In another aspect, the second amplification oligomer pair comprises a first amplification oligomer comprising, consisting or consisting essentially of a target binding sequence as set forth in any one of SEQ ID NOS: 105 to 109 and a second amplification oligomer comprising, consisting or consisting essentially of a target binding sequence as set forth in any one of SEQ ID NOS: 128 to 133. In another aspect, the first amplification oligomer pair is one of SEQ ID NOS: 97 to 104 and one of SEQ ID NOS: 110 to 115 or 122 to 127. In another aspect, the second amplification oligomer pair is one of SEQ ID NOS: 105 to 109 and one of SEQ ID NOS: 116 to 121 or 128 to 133.

In one aspect, the composition or reaction mixture further includes a detection probe oligomer wherein said detection probe oligomer comprises a target binding sequence set forth in SEQ ID NO: 148 to 150. In another aspect, the composition or reaction mixture further includes a detection probe oligomer wherein said detection probe oligomer comprises a target binding sequence set forth in SEQ ID NO: 151 to 156.

Compositions can be included in a kit. One embodiment is a kit containing one of the compositions described herein. In one aspect, the kit is for use with screening blood. In one aspect, the kit is for screening blood used for blood banking. In one aspect, the kit is for screening blood for blood transfusions. In one embodiment, there is provided a reaction mixture containing one or more compositions for use in any one of the method steps described herein. Reaction mixtures can contain one or more of the compositions described herein, including amplification oligomers, target capture oligomers, detection probe oligomers and amplification products.

One embodiment includes compositions, kits, reaction mixtures and amplification and/or detection methods that use one or more oligonucleotides from the Sequence Listing as a primer or a probe. In one aspect, the compositions specifically hybridize to a murine leukemia virus related virus. In one aspect, one or more of the compositions are packaged in a kit. In one aspect the packaged kit includes instructions for use of the compositions in a method for the amplification and/or detection of a murine leukemia virus related virus. In one aspect, one or more of the compositions are used in a reaction mixture. In one aspect, the reaction mixture is a target capture reaction mixture, an amplification reaction mixture, a detection reaction mixture or a combination thereof. In one aspect, one or more of the compositions are used in a method for the amplification of a murine leukemia virus related virus. In one aspect, one or more of the compositions are used in a method for the detection of a murine leukemia virus related virus. In one aspect, an amplicon containing a sequence from the Sequence Listing is provided.

### DETAILED DESCRIPTION OF THE INVENTION

To aid in understanding aspects of the disclosure, some terms used herein are described in more detail. All other scientific and technical terms used herein have the same meaning as commonly understood by those skilled in the relevant art, such as may be provided in Dictionary of Microbiology and Molecular Biology, 2nd ed. (Singleton et al., 1994, John Wiley & Sons, New York, N.Y.), The Harper Collins Dictionary of Biology (Hale & Marham, 1991, Harper Perennial, New York, N.Y.), and references cited herein. Unless mentioned otherwise, the techniques employed or contemplated herein are standard methods well known to a person of ordinary skill in the art of molecular biology.

It is to be noted that the term “a” or “an” entity refers to one or more of that entity; for example, “a nucleic acid,” is understood to represent one or more nucleic acids. As such, the terms “a” (or “an”), “one or more,” and “at least one” can be used interchangeably herein.

Sample. A “sample” or “specimen”, including “biological” or “clinical” samples may contain or may be suspected of containing MLV or components thereof, such as nucleic acids or fragments of nucleic acids. A sample may be a complex mixture of components. Samples include “biological samples” which include any tissue or material derived from a living or dead mammal or organism, including, e.g., urine, prostatic secretions and/or fluids, semen, blood, plasma, serum, blood cells, saliva, and mucous, cerebrospinal fluid, and other samples—such as biopsies—from or derived from a tissue sample (e.g., a tissue sample from or derived from genital lesions, anogenital lesions, oral lesions, mucocutanoeus lesions, skin lesions and ocular lesions prostate, bladder, seminal glands, testes, kidney, bone marrow, adrenal glands, liver, heart, lung, colon, ileum, jejunum, pancreas, spleen, brain cortex, brain stem, cerebellum, axillar lymph node inguinal lymph node and/or mesenteric lymph node), a tumour sample (e.g., a prostate tumour or a bladder tumour, or another tumours of the male or female genitourinary tracts) and combinations thereof. Samples may also include samples of in vitro cell culture constituents including, eg., conditioned media resulting from the growth of cells and tissues in culture medium. The sample may be treated to physically or mechanically disrupt tissue or cell structure to release intracellular nucleic acids into a solution which may contain enzymes, buffers, salts, detergents and the like, to prepare the sample for analysis. In one step of the methods described herein, a sample is provided that is suspected of containing at least a MLV target nucleic acid. Accordingly, this step excludes the physical step of obtaining the sample from a subject.

Nucleic acid. The term “nucleic acid” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid “backbone” may be made up of a variety of linkages, including one or more of sugar-phosphodiester linkages, peptide-nucleic acid bonds (in “peptide nucleic acids” or PNAs, see PCT No. WO 95/32305), phosphorothioate linkages, methylphosphonate linkages, or combinations thereof. Sugar moieties of the nucleic acid may be either ribose or deoxyribose, or similar compounds having known substitutions, e.g., 2′ methoxy substitutions and 2′ halide substitutions (e.g., 2′-F). Nitrogenous bases may be conventional bases (A, G, C, T, U), analogs thereof (e.g., inosine, 5-methylisocytosine, isoguanine; The Biochemistry of the Nucleic Acids 5-36, Adams et al., ed., 11^{th} ed., 1992, Abraham et al., 2007, BioTechniques 43: 617-24), which include derivatives of purine or pyrimidine bases (e.g., N⁴-methyl deoxygaunosine, deaza- or aza-purines, deaza- or aza-pyrimidines, pyrimidine bases having substituent groups at the 5 or 6 position, purine bases having an altered or replacement substituent at the 2, 6 and/or 8 position, such as 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines, and pyrazolo-compounds, such as unsubstituted or 3-substituted pyrazolo[3,4-d]pyrimidine; U.S. Pat. Nos. 5,378,825, 6,949,367 and PCT No. WO 93/13121). Nucleic acids may include “abasic” residues in which the backbone does not include a nitrogenous base for one or more residues (U.S. Pat. No. 5,585,481). A nucleic acid may comprise only conventional sugars, bases, and linkages as found in RNA and DNA, or may include conventional components and substitutions (e.g., conventional bases linked by a 2′ methoxy backbone, or a nucleic acid including a mixture of conventional bases and one or more base analogs). Nucleic acids may include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA) (Vester et al., 2004, Biochemistry 43(42):13233-41). Nucleic acids may include modified bases to alter the function or behavior of the nucleic acid, e.g., addition of a 3′-terminal dideoxynucleotide to block additional nucleotides from being added to the nucleic acid. Synthetic methods for making nucleic acids in vitro are well known in the art although nucleic acids may be purified from natural sources using routine techniques.

Polynucleotide. The term “polynucleotide” denotes a nucleic acid chain. Throughout this application, nucleic acids are designated by the 5′-terminus to the 3′-terminus. Standard nucleic acids, e.g., DNA and RNA, are typically synthesized “3′-to-5′,” i.e., by the addition of nucleotides to the 5′-terminus of a growing nucleic acid.

Nucleotide. As referred to herein, a “nucleotide” is a subunit of a nucleic acid consisting of a phosphate group, a 5-carbon sugar and a nitrogenous base. The 5-carbon sugar found in RNA is ribose. In DNA, the 5-carbon sugar is 2′-deoxyribose. The term also includes analogs of such subunits, such as a methoxy group at the 2′ position of the ribose (2′-O-Me, or 2′ methoxy). As used herein, methoxy oligonucleotides containing “T” residues have a methoxy group at the 2′ position of the ribose moiety, and a uracil at the base position of the nucleotide.

Non-nucleotide unit. The term “non-nucleotide unit” is a unit that does not significantly participate in hybridization of a polymer. Such units must not, for example, participate in any significant hydrogen bonding with a nucleotide, and would exclude units having as a component one of the five nucleotide bases or analogs thereof.

Target nucleic acid. The term “target nucleic acid” refers to a nucleic acid comprising a “target sequence” to be amplified. Target nucleic acids may be DNA or RNA and may be either single-stranded or double-stranded. In one embodiment, the target nucleic acid is RNA. In another embodiment, the target nucleic acid is an amplification product that has not been obtained by reverse transcription of nucleic acid. In another embodiment, the target nucleic acid is RNA that is from or is derived from MLV. In another embodiment, the target nucleic acid is RNA encoded by the DNA sequence set forth in SEQ ID No. 85. In another embodiment, the target nucleic acid is RNA that is from or is derived from GenBank Accession No. DQ241301, GenBank Accession No. DQ241302, or GenBank Accession No. DQ399707. In another aspect, the target nucleic acid is a nucleic acid comprising a target sequence selected from the group consisting of: from residue 2800 to residue 2862 of SEQ ID NO:85; residue 2924 to residue 2971 of SEQ ID NO:85; from residue 7676 to residue 7713 of SEQ ID NO:85, from residue 7756 to residue 7804 of SEQ ID NO:85; and combinations thereof. The target nucleic acid may include other sequences besides the target sequence that may be amplified. Typical target nucleic acids include virus genomes, bacterial genomes, fungal genomes, plant genomes, animal genomes, rRNA, tRNA, or mRNA from viruses, bacteria or eukaryotic cells, mitochondrial DNA, or chromosomal DNA. In the instant disclosure, target nucleic acids are nucleic acids from MLV, or amplification products thereof. In one aspect, the target nucleic acid is RNA from MLV. In another aspect, the target nucleic acid is an amplification product generated from an MLV nucleic acid. The amplification product can be generated using any amplification method; PCR and TMA being two non-limiting examples. The amplification product target nucleic acid can be either single stranded or double stranded. Double stranded target nucleic acids can be DNA:DNA, DNA:RNA or RNA:RNA.

Target sequence. The term “target sequence” refers to the particular nucleotide sequence of the target nucleic acid that is to be amplified. In one aspect, the target sequence is selected from the group consisting of: from residue 2800 to residue 2862 of SEQ ID NO:85; residue 2924 to residue 2971 of SEQ ID NO:85; from residue 7676 to residue 7713 of SEQ ID NO:85, from residue 7756 to residue 7804 of SEQ ID NO:85; and combinations thereof. Where the target nucleic acid is originally single-stranded, the term “target sequence” will also refer to the sequence complementary to the target sequence as present in the target nucleic acid. Where the target nucleic acid is originally double-stranded, the term “target sequence” refers to both the sense (+) and antisense (−) strands. In choosing a target sequence, the skilled artisan will understand that a sequence should be chosen so as to distinguish between unrelated or closely related target nucleic acids.

The terms “target(s) a sequence” or “target(s) a target nucleic acid” as used herein in reference to a region of MLV nucleic acid refers to a process whereby an oligonucleotide stably hybridizes to the referenced sequence in a manner that allows for amplification and/or detection as described herein. In one embodiment, the oligonucleotide is complementary to the targeted MLV nucleic acid sequence and contains no mismatches. In another embodiment, the oligonucleotide is complementary but contains 1; or 2; or 3; or 4; or 5; or 6; or 7; or 8; or 9; or 10 or more mismatches with the targeted MLV nucleic acid sequence. Preferably, the oligonucleotide that stably hybridizes to the MLV nucleic acid sequence includes at least 10, 11, 12, 13, 14, 15, 16, 17, 18, 19, 20, 21, 22, 23, 24, 25, 30, 35, 40, 45 or 50 nucleotides complementary to the target sequence. It is understood that at least 10 and as many as 50 is an inclusive range such that 10, 50 and each whole number there between are included. The term “configured to target a sequence” as used herein means that the target hybridizing region of an amplification oligonucleotide is designed to have a polynucleotide sequence that could target a sequence of the referenced MLV region. Such an amplification oligonucleotide is not limited to targeting that sequence only, but is rather useful as a composition, in a kit or in a method for targeting a MLV target nucleic acid, as is described herein. The term “configured to” denotes an actual arrangement of the polynucleotide sequence configuration of the amplification oligonucleotide target hybridizing sequence.

Isolated. The term “isolated” means that a nucleic acid is taken from its natural milieu, but the term does not connote any degree of purification.

Fragment. This term as used herein in reference to the MLV targeted nucleic acid sequence refers to a piece of contiguous nucleic acid. In certain embodiments, the fragment includes contiguous nucleotides from an MLV target nucleic acid, wherein the number of contiguous nucleotides in the fragment are less than that for the entire POL gene or LTR gene.

Region. The term “region” refers to a portion of a nucleic acid wherein said portion is smaller than the entire nucleic acid. For example, when the nucleic acid in reference is an oligonucleotide promoter provider, the term “region” may be used refer to the smaller promoter portion of the entire oligonucleotide. Similarly, and also as example only, when the referenced nucleic acid is a target nucleic acid, the term “region” may be used to refer to a smaller area of the nucleic acid.

Oligonucleotide. “Oligonucleotide” may be used interchangeably with “oligomer and “oligo” and refers to a nucleic acid having generally more than 5 nucleotide (nt) residues, and less than 1,000 nucleotide (nt) residues, such as from about 5 nt residues to about 900 nt residues, from about 10 nt residues to about 800 nt residues with a lower limit of about 12 to 15 nt and an upper limit of about 40 to 600 nt, and other embodiments are in a range having a lower limit of about 15 to 20 nt and an upper limit of about 22 to 100 nt. This range includes all encompassed whole numbers. It is understood that this range is exemplary only. Oligonucleotides may be purified from naturally occurring sources, or may be synthesized using any of a variety of well known enzymatic or chemical methods. The term oligonucleotide does not denote any particular function to the reagent; rather, it is used generically to cover all such reagents described herein. An oligonucleotide may serve various different functions. For example, it may function as a primer if it is specific for and capable of hybridizing to a complementary strand and can further be extended in the presence of a nucleic acid polymerase, it may provide a promoter if it contains a sequence recognized by an RNA polymerase and allows for transcription (eg., a T7 provider), and it may function to prevent hybridization or impede primer extension if appropriately situated and/or modified.

As used herein, an oligonucleotide having a nucleic acid sequence “comprising” or “consisting of” or “consisting essentially of” a sequence selected from a group of specific sequences means that the oligonucleotide, as a basic and novel characteristic, is capable of stably hybridizing to a nucleic acid having the exact complement of one of the listed nucleic acid sequences of the group under stringent hybridization conditions. An exact complement includes the corresponding DNA or RNA sequence.

Corresponds. As used herein, a nucleic acid “corresponds” to a specified nucleic acid if the nucleic acid is 100% identical or complementary to the specified nucleic acid.

Substantially corresponding to. As used herein, a nucleic acid “substantially corresponding to” a specified nucleic acid sequence means that the referred to oligonucleotide is sufficiently similar to the reference nucleic acid sequence such that the oligonucleotide has similar hybridization properties to the reference nucleic acid sequence in that it would hybridize with the same target nucleic acid sequence under stringent hybridization conditions. Substantially corresponding nucleic acids vary by at least one nucleotide from the specified nucleic acid. This variation may be stated in terms of a percentage of identity or complementarity between the nucleic acid and the specified nucleic acid. Thus, nucleic acid substantially corresponds to a reference nucleic acid sequence if these percentages of base identity or complementarity are from less than 100% to about 80%. In preferred embodiments, the percentage is at least about 85%. In more preferred embodiments, this percentage is at least about 90%; in other preferred embodiments, this percentage is at least about 95%, 96%, 97%, 98% or 99%. One skilled in the art will understand that the recited ranges include all whole and rational numbers of the range (e.g., 92% or 92.377%).

Blocking moiety. As used herein, a “blocking moiety” is a substance used to “block” the 3′-terminus of an oligonucleotide or other nucleic acid so that it cannot be efficiently extended by a nucleic acid polymerase.

Amplification oligomer. An “amplification oligomer”, which may also be called an “amplification oligonucleotide” is an oligomer, at least the 3′-end of which is complementary to a target nucleic acid (“target hybridizing sequence” or “target binding sequence” OR “target binding region”), and which hybridizes to a target nucleic acid, or its complement, and participates in a nucleic acid amplification reaction. An example of an amplification oligomer is a “primer” that hybridizes to a target nucleic acid and contains a 3′ OH end that is extended by a polymerase in an amplification process. Another example of an amplification oligomer is a “promoter-based amplification oligomer,” which comprises a target hybridizing sequence, and a promoter sequence for initiating transcription by an appropriate polymerase. Promoter-based amplification oligomers may or may not be extended by a polymerase in a primer-based extension depending upon whether or not the 3′ end of the target hybridizing sequence is modified to prevent primer-based extension (e.g., a 3′ blocked end). A promoter-based amplification oligonucleotide comprising a target hybridizing region that is not modified to prevent primer-based extension is referred to as a “promoter-primer.” A promoter-based amplification oligonucleotide comprising a target hybridizing region that is modified to prevent primer-based extension is referred to as a “promoter-provider.” Size ranges for amplification oligonucleotides include those comprising target hybridizing regions that are about 10 to about 70 nt long—such as about 10 to about 60 nt long, about 10 to about 50 nt long, about 10 to about 40 nt long, about 10 to about 30 nt long or about 10 to about 25 nt long or about 15 to 25 nt long. Preferred sizes of amplification oligomers include those comprising target hybridizing regions that are about 18, 19, 20, 21, 22 or 23 nt long. An amplification oligomer may optionally include modified nucleotides or analogs that are not complementary to target nucleic acid in a strict A:T/U, G:C sense. Such modified nucleotides or analogs are herein considered mismatched to their corresponding target sequence. For some embodiments, the preferred amount of amplification oligomer per reaction is about 10, 15 or 20 pmoles.

Oligomers not intended for primer-based extension by a nucleic acid polymerase may include a blocker group that replaces the 3′ H to prevent the enzyme-mediated extension of the oligomer in an amplification reaction. For example, blocked amplification oligomers and/or detection probes present during amplification may not have functional 3′ H and instead include one or more blocking groups located at or near the 3′ end. In some embodiments a blocking group near the 3′ end and may be within five residues of the 3′ end and is sufficiently large to limit binding of a polymerase to the oligomer. In other embodiments a blocking group is covalently attached to the 3′ terminus. Many different chemical groups may be used to block the 3′ end, e.g., alkyl groups, non-nucleotide linkers, alkane-diol dideoxynucleotide residues, and cordycepin.

Promoter. The term “promoter” refers to a specific nucleic acid sequence that is recognized by a DNA-dependent RNA polymerase (“transcriptase”) as a signal to bind to the nucleic acid and begin the transcription of RNA at a specific site. Promoters include, SP6 promoters, T3 promoters and T7 promoters, to name a few.

Promoter-provider. As used herein, a “promoter-provider” or “provider” refers to an oligonucleotide comprising first and second regions, and which is modified to prevent the initiation of DNA synthesis from its 3′-terminus. The “first region” of a promoter-provider oligonucleotide comprises a base sequence which hybridizes to a DNA template, where the hybridizing sequence is situated 3′, but not necessarily adjacent to, a promoter region. The target-hybridizing portion of a promoter oligonucleotide is typically at least 10, 11, 12, 13, 14, 15, 16, 17, 18, 19, 20, 21, 22, 23, 24, 25, 30, 35, 40 or 45 nucleotides in length, and may extend up to 50 or more nucleotides in length. The “second region” comprises a promoter sequence for an RNA polymerase. A promoter-provider oligonucleotide is configured so that it is incapable of being extended by an RNA- or DNA-dependent DNA polymerase, (e.g., reverse transcriptase), preferably by comprising a blocking moiety at its 3′-terminus as described above. This modification differentiates promoter providers from promoter primers. Preferably, the promoter portion of a promoter primer or provider is a promoter for a DNA-dependent RNA polymerase from E. coli and bacteriophages T7, T3, and SP6, though other promoters or modified version thereof can be used as well.

Terminating oligonucleotide. As used herein, a “terminating oligonucleotide” or “blocker oligonucleotide” is an oligonucleotide comprising a base sequence that is complementary to a region of the target nucleic acid in the vicinity of the 5′-end of the target sequence, so as to “terminate” primer extension of a nascent nucleic acid that includes a priming oligonucleotide, thereby providing a defined 3′-end for the nascent nucleic acid strand.

Amplification. This refers to any known procedure for obtaining multiple copies of a target nucleic acid sequence or its complement or fragments thereof. The multiple copies may be referred to as amplicons or amplification products. Amplification of “fragments” refers to production of an amplified nucleic acid that contains less than the complete target nucleic acid or its complement, eg., produced by using an amplification oligonucleotide that hybridizes to, and initiates polymerization from, an internal position of the target nucleic acid. Known amplification methods include both thermal cycling and isothermal amplification methods. For some embodiment, isothermal amplification methods are preferred. Replicase-mediated amplification, polymerase chain reaction (PCR), ligase chain reaction (LCR), strand-displacement amplification (SDA), and transcription-mediated or transcription-associated amplification are non-limiting examples of nucleic acid amplification methods. Replicase-mediated amplification uses self-replicating RNA molecules, and a replicase such as QB-replicase (eg., U.S. Pat. No. 4,786,600). PCR amplification uses a DNA polymerase, pairs of primers, and thermal cycling to synthesize multiple copies of two complementary strands of dsDNA or from a cDNA (eg., U.S. Pat. Nos. 4,683,195, 4,683,202, and 4,800,159). LCR amplification uses four or more different oligonucleotides to amplify a target and its complementary strand by using multiple cycles of hybridization, ligation, and denaturation (eg., U.S. Pat. No. 5,427,930 and U.S. Pat. No. 5,516,663). SDA uses a primer that contains a recognition site for a restriction endonuclease and an endonuclease that nicks one strand of a hemimodified DNA duplex that includes the target sequence, whereby amplification occurs in a series of primer extension and strand displacement steps (eg., U.S. Pat. No. 5,422,252; U.S. Pat. No. 5,547,861; and U.S. Pat. No. 5,648,211). Preferred embodiments use an amplification method suitable for the amplification of RNA target nucleic acids, such as transcription mediated amplification (TMA) or NASBA, but it will be apparent to persons of ordinary skill in the art that oligomers disclosed herein may be readily used as primers in other amplification methods.

Transcription associated amplification. This method of amplification, also referred to herein as “transcription mediated amplification” (TMA) refers to nucleic acid amplification that uses an RNA polymerase to produce multiple RNA transcripts from a nucleic acid template. These methods generally employ an RNA polymerase, a DNA polymerase, deoxyribonucleoside triphosphates, ribonucleoside triphosphates, and a template complementary oligonucleotide that includes a promoter sequence, and optionally may include one or more other oligonucleotides. TMA methods are embodiments of amplification methods used for amplifying and detecting MLV target sequences as described herein. Variations of transcription associated amplification are well known in the art as previously disclosed in detail (eg., U.S. Pat. Nos. 4,868,105; 5,124,246; 5,130,238; 5,399,491; 5,437,990; 5,554,516; and 7,374,885; and PCT Pub. Nos. WO 88/01302; WO 88/10315 and WO 95/03430). The person of ordinary skill in the art will appreciate that the disclosed compositions may be used in amplification methods based on extension of oligomer sequences by a polymerase.

Real-time TMA. As used herein, the term “real-time TMA” refers to single-primer transcription-mediated amplification (“TMA”) of target nucleic acid that is monitored by real-time detection means.

Amplicon. This term, which is used interchangeably with “amplification product”, refers to the nucleic acid molecule generated during an amplification procedure that is complementary or homologous to a sequence contained within the target sequence. These terms can be used to refer to a single strand amplification product, a double strand amplification product or one of the strands of a double strand amplification product.

Probe. A probe, also known as a “detection probe” or “detection oligonucleotide” are terms referring to a nucleic acid oligomer that hybridizes specifically to a target sequence in a nucleic acid, or in an amplified nucleic acid, under conditions that promote hybridization to allow detection of the target sequence or amplified nucleic acid. Probes may be DNA, RNA, analogs thereof or combinations thereof and they may be labeled or unlabeled. Detection may either be direct (e.g., a probe is hybridized directly to specifically hybridize to a smaller nucleic acid sequence within a larger target sequence) or indirect (e.g., a probe is linked to its target via an intermediate molecular structure). A probe is generally configured to specifically hybridize to a smaller nucleic acid sequence within a larger target sequence by standard base pairing. A probe may comprise target-specific sequences and other sequences that contribute to the three-dimensional conformation of the probe (e.g., U.S. Pat. Nos. 5,118,801; 5,312,728; 6,849,412; 6,835,542; 6,534,274; and 6,361,945; and US Pub. No. 20060068417). Exemplary probe types include, nucleic acid probes, AE-labeled nucleic acid probes, molecular beacons, molecular torches, molecular switches, taqman probes, hairpin probes, and other well-known configurations. In a preferred embodiment, the detection probe comprises a 2′ methoxy backbone which can result in a higher signal being obtained.

Molecular torches. As used herein, structures referred to as “molecular torches” are designed to include distinct regions of self-complementarity (“the closing domain”) which are connected by a joining region (“the target binding domain”) and which hybridize to one another under predetermined hybridization assay conditions. All or part of the nucleotide sequences comprising target closing domains may also function as target binding domains. Thus, target closing sequences can include, target binding sequences, non-target binding sequences, and combinations thereof.

Stable. By “stable” or “stable for detection” is meant that the temperature of a reaction mixture is at least 2.deg. C. below the melting temperature of a nucleic acid duplex.

Label. As used herein, a “label” refers to a moiety or compound joined directly or indirectly to a probe that is detected or leads to a detectable signal. Direct labelling can occur through bonds or interactions that link the label to the probe, including covalent bonds or non-covalent interactions, e.g. hydrogen bonds, hydrophobic and ionic interactions, or formation of chelates or coordination complexes. Indirect labelling can occur through use of a bridging moiety or “linker” such as a binding pair member, an antibody or additional oligomer, which is either directly or indirectly labeled, and which may amplify the detectable signal. Labels include any detectable moiety, such as a radionuclide, ligand (e.g., biotin, avidin), enzyme or enzyme substrate, reactive group, or chromophore (e.g., dye, particle, or bead that imparts detectable color), luminescent compound (e.g., bioluminescent, phosphorescent, or chemiluminescent labels), chemiluminescent compounds, e.g., acridinium ester (“AE”) compounds that include standard AE and derivatives (e.g., U.S. Pat. Nos. 5,656,207, 5,658,737, and 5,639,604), quencher or fluorophore. Labels may be detectable in a homogeneous assay in which bound labeled probe in a mixture exhibits a detectable change different from that of an unbound labeled probe, e.g., instability or differential degradation properties. A “homogeneous detectable label” can be detected without physically removing bound from unbound forms of the label or labeled probe (e.g., U.S. Pat. Nos. 5,283,174, 5,656,207, and 5,658,737). Synthesis and methods of attaching labels to nucleic acids and detecting labels are well known (e.g., Sambrook et al., Molecular Cloning, A Laboratory Manual, 2nd ed. (Cold Spring Harbor Laboratory Press, Cold Spring Harbor, N.Y., 1989), Chapter 10; U.S. Pat. Nos. 5,658,737, 5,656,207, 5,547,842, 5,283,174, and 4,581,333). More than one label, and more than one type of label, may be present on a particular probe, or detection may use a mixture of probes in which each probe is labeled with a compound that produces a detectable signal (e.g., U.S. Pat. Nos. 6,180,340 and 6,350,579).

Capture oligonucleotide. As used herein, a “capture oligonucleotide,” “target capture oligonucleotide” or “capture probe” refers to a nucleic acid oligomer that specifically hybridizes to a target sequence in a target nucleic acid by standard base pairing and joins to a binding partner on an immobilized probe to capture the target nucleic acid to a support. One example of a capture oligomer includes an oligonucleotide comprising two binding regions: a target hybridizing sequence and an immobilized probe-binding region. A variation of this example, the two regions may be present on two different oligomers joined together by one or more linkers. Another embodiment of a capture oligomer the target hybridizing sequence is a sequence that includes random or non-random poly-GU, poly-GT, or poly U sequences to bind non-specifically to a target nucleic acid and link it to an immobilized probe on a support. (PCT Pub No. WO 2008/016988). The immobilized probe binding region can be a nucleic acid sequence, referred to as a tail. Tails include a substantially homopolymeric tail (T₀₋₄A₁₀₋₄₀), that bind to a complementary immobilized sequence attached to the support particle or support matrix. Thus, a non-limiting example of preferred nucleic acid tails can in some embodiments include about 10 to 40 nucleotides (e.g., A₁₀ to A₄₀), or of about 14 to 33 nt (e.g., T₃A₁₄ to T₃A₃₀). Another example of a capture oligomer comprises two regions, a target hybridizing sequence and a binding pair member that is not a nucleic acid sequence.

Immobilized oligonucleotide. As used herein, an “immobilized oligonucleotide”, “immobilized probe” or “immobilized nucleic acid” refers to a nucleic acid binding partner that joins a capture oligomer to a support, directly or indirectly. An immobilized probe joined to a support facilitates separation of a capture probe bound target from unbound material in a sample. One embodiment of an immobilized probe is an oligomer joined to a support that facilitates separation of bound target sequence from unbound material in a sample. Supports may include known materials, such as matrices and particles free in solution, which may be made of nitrocellulose, nylon, glass, polyacrylate, mixed polymers, polystyrene, silane, polypropylene, metal, or other compositions, of which one embodiment is magnetically attractable particles. Supports may be monodisperse magnetic spheres (e.g., uniform size±5%), to which an immobilized probe is joined directly (via covalent linkage, chelation, or ionic interaction), or indirectly (via one or more linkers), where the linkage or interaction between the probe and support is stable during hybridization conditions.

Complementary. By “complementary” is meant that the nucleotide sequences of similar regions of two single-stranded nucleic acids, or to different regions of the same single-stranded nucleic acid have a nucleotide base composition that allow the single-stranded regions to hybridize together in a stable double-stranded hydrogen-bonded region under stringent hybridization or amplification conditions. Sequences that hybridize to each other may be completely complementary or partially complementary to the intended target sequence by standard nucleic acid base pairing (e.g. G:C, A:T or A:U pairing). By “sufficiently complementary” is meant a contiguous sequence that is capable of hybridizing to another sequence by hydrogen bonding between a series of complementary bases, which may be complementary at each position in the sequence by standard base pairing or may contain one or more residues that are not complementary by standard A:T/U and G:C pairing, or are modified nucleotides such as abasic residues, modified nucleotides or nucleotide analogs. Sufficiently complementary contiguous sequences typically are at least 80%, or at least 90%, complementary to a sequence to which an oligomer is intended to specifically hybridize (a %-complementarity range includes all whole and rational numbers of the range). Sequences that are “sufficiently complementary” allow stable hybridization of a nucleic acid oligomer with its target sequence under appropriate hybridization conditions, even if the sequences are not completely complementary. When a contiguous sequence of nucleotides of one single-stranded region is able to form a series of “canonical” hydrogen-bonded base pairs with an analogous sequence of nucleotides of the other single-stranded region, such that A is paired with U or T and C is paired with G, the nucleotides sequences are “completely” complementary.

Preferentially hybridize. By “preferentially hybridize” is meant that under stringent hybridization assay conditions, an oligonucleotide hybridizes to its target sequences, or replicates thereof, to form stable oligonucleotide: target sequence hybrid, while at the same time formation of stable oligonucleotide: non-target sequence hybrid is minimized. For example, a probe oligonucleotide preferentially hybridizes to a target sequence or replicate thereof to a sufficiently greater extent than to a non-target sequence, to enable one having ordinary skill in the art to accurately detect the RNA replicates or complementary DNA (cDNA) of the target sequence formed during the amplification. Appropriate hybridization conditions are well known in the art for probe, amplification, target capture, blocker and other oligonucleotides, may be predicted based on sequence composition, or can be determined by using routine testing methods (e.g., Sambrook et al., Molecular Cloning, A Laboratory Manual, 2^{nd} ed. (Cold Spring Harbor Laboratory Press, Cold Spring Harbor, N.Y., 1989) at §§1.90-1.91, 7.37-7.57, 9.47-9.51 and 11.47-11.57, particularly §§9.50-9.51, 11.12-11.13, 11.45-11.47 and 11.55-11.57).

Nucleic acid hybrid. By “nucleic acid hybrid” or “hybrid” or “duplex” is meant a nucleic acid structure containing a double-stranded, hydrogen-bonded region wherein each strand is complementary to the other, and wherein the region is sufficiently stable under stringent hybridization conditions to be detected by means including, but not limited to, chemiluminescent or fluorescent light detection, autoradiography, or gel electrophoresis. Such hybrids may comprise RNA:RNA, RNA:DNA, or DNA:DNA duplex molecules.

Sample preparation. This refers to any steps or methods that treat a sample for subsequent amplification and/or detection of MLV nucleic acids present in the sample. The target nucleic acid may be a minority component in the sample. Sample preparation may include any known method of isolating or concentrating components, such as viruses or nucleic acids using standard microbiology methods. Sample preparation may include physical disruption and/or chemical lysis of cellular components to release intracellular components into a substantially aqueous or organic phase and removal of debris, such as by using filtration, centrifugation or adsorption. Sample preparation may include use of a nucleic acid oligonucleotide that selectively or non-specifically captures a target nucleic acid and separates it from other sample components (eg., as described in U.S. Pat. No. 6,110,678 and PCT Pub. No. WO 2008/016988).

Separating, purifying. These terms mean that one or more components of a sample are removed or separated from other sample components. Sample components include target nucleic acids usually in a generally aqueous solution phase, which may also include cellular fragments, proteins, carbohydrates, lipids, and other nucleic acids. Separating or purifying removes at least 70%, or at least 80%, or at least 95% of the target nucleic acid from other sample components. Ranges of %-purity include all whole and rational numbers of the range.

DNA-dependent DNA polymerase. As used herein, a “DNA-dependent DNA polymerase” is an enzyme that synthesizes a complementary DNA copy from a DNA template. Examples are DNA polymerase I from E. coli, bacteriophage T7 DNA polymerase, or DNA polymerases from bacteriophages T4, Phi-29, M2, or T5. DNA-dependent DNA polymerases may be the naturally occurring enzymes isolated from bacteria or bacteriophages or expressed recombinantly, or may be modified or “evolved” forms which have been engineered to possess certain desirable characteristics, e.g., thermostability, or the ability to recognize or synthesize a DNA strand from various modified templates. All known DNA-dependent DNA polymerases require a complementary primer to initiate synthesis. It is known that under suitable conditions a DNA-dependent DNA polymerase may synthesize a complementary DNA copy from an RNA template. RNA-dependent DNA polymerases typically also have DNA-dependent DNA polymerase activity.

DNA-dependent RNA polymerase. As used herein, a “DNA-dependent RNA polymerase” or “transcriptase” is an enzyme that synthesizes multiple RNA copies from a double-stranded or partially double-stranded DNA molecule having a promoter sequence that is usually double-stranded. The RNA molecules (“transcripts”) are synthesized in the 5′-to-3′ direction beginning at a specific position just downstream of the promoter. Examples of transcriptases are the DNA-dependent RNA polymerase from E. coli and bacteriophages T7, T3, and SP6.

RNA-dependent DNA polymerase. As used herein, an “RNA-dependent DNA polymerase” or “reverse transcriptase” (“RT”) is an enzyme that synthesizes a complementary DNA copy from an RNA template. All known reverse transcriptases also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

Selective RNAse. As used herein, a “selective RNAse” is an enzyme that degrades the RNA portion of an RNA:DNA duplex but not single-stranded RNA, double-stranded RNA or DNA. An exemplary selective RNAse is RNAse H. Enzymes possessing the same or similar activity as RNAse H may also be used. Selective RNAses may be endonucleases or exonucleases. Most reverse transcriptase enzymes contain an RNAse H activity in addition to their polymerase activities. However, other sources of the RNAse H are available without an associated polymerase activity. The degradation may result in separation of RNA from a RNA:DNA complex. Alternatively, a selective RNAse may simply cut the RNA at various locations such that portions of the RNA melt off or permit enzymes to unwind portions of the RNA. Other enzymes that selectively degrade RNA target sequences or RNA products of the present invention will be readily apparent to those of ordinary skill in the art.

Specificity. The term “specificity,” in the context of an amplification system, is used herein to refer to the characteristic of an amplification system which describes its ability to distinguish between target and non-target sequences dependent on sequence and assay conditions. In terms of nucleic acid amplification, specificity generally refers to the ratio of the number of specific amplicons produced to the number of side-products (e.g., the signal-to-noise ratio).

Sensitivity. The term “sensitivity” is used herein to refer to the precision with which a nucleic acid amplification reaction can be detected or quantitated. The sensitivity of an amplification reaction is generally a measure of the smallest copy number of the target nucleic acid that can be reliably detected in the amplification system, and will depend, for example, on the detection assay being employed, and the specificity of the amplification reaction, e.g., the ratio of specific amplicons to side-products.

Relative fluorescence unit. As used herein, the term “relative fluorescence unit” (“RFU”) is an arbitrary unit of measurement of fluorescence intensity. RFU varies with the characteristics of the detection means used for the measurement.

Helper oligonucleotide. A “helper oligonucleotide” or “helper” refers to an oligonucleotide designed to bind to a target nucleic acid and impose a different secondary and/or tertiary structure on the target to increase the rate and extent of hybridization of a detection probe or other oligonucleotide with the targeted nucleic acid, as described, for example, in U.S. Pat. No. 5,030,557. Helpers may also be used to assist with the hybridization to target nucleic acid sequences and function of primer, target capture and other oligonucleotides. Helper oligonucleotides may be used in the methods described herein and may form part of the compositions and kits described herein.

### Oligonucleotides for the Amplification and Detection of MLV

In one embodiment, an amplification reaction is performed using at least one pair of amplification oligomers. In another embodiment, two or more pairs of amplification oligomers are used for multiplex amplification reactions. Amplification oligomers comprise target binding sequences. In some aspects, the target binding sequences are optionally combined with an additional nucleic acid region(s). In-some aspects, the additional region(s) of nucleic acids are arranged 5′ to the target binding sequences. In some aspects, the oligomers described herein comprise additional sequences at their 5′ and/or 3′ ends which may or may not be complementary to MLV nucleic acid.

One or more of the target binding regions making up the amplification oligomers, include those that are from about 10 to about 50 nucleobases in length and are configured to specifically hybridize to a region within a target sequence of MLV, wherein said region is from residue 2800 to residue 2862, or from 2924 to residue 2971, or from residue 7676 to residue 7713, or from residue 7756 to residue 7804 of SEQ ID NO:85 (GenBank Accession Number EF185282.1 GI:121104176 entered at NCBI on Jan. 10, 2007). Exemplary target binding sequences include those that contain, comprise, consist or consist essentially of SEQ ID NOS: 99, 102, 103, 109, 125, 127, 130, 131, 157, 158 or 159. Exemplary target binding sequences include those comprising, consisting or consisting essentially of SEQ ID NOS:97-109 and 122-133. Exemplary amplification oligomer pairs include those configured to generate an amplicon of about 200 nucleobases from a target nucleic acid. Exemplary multiplex amplification oligomers include those configured to generate at least two amplification products each independently being of about 200 nucleobases or less in length, wherein at least one contains, comprises, consists or consists essentially of a nucleotide sequence corresponding to residues 2862 to 2924 of SEQ ID NO:85, or wherein at least one contains, comprises, consists or consists essentially of a nucleotide sequence corresponding to residues 7713 to 7756 of SEQ ID NO:85, or wherein one contains, comprises, consists or consists essentially of a nucleotide sequence corresponding to residues 2862 to 2924 of SEQ ID NO:85 and one contains, comprises, consists or consists essentially of a nucleotide sequence corresponding to residues 7713 to 7756 of SEQ ID NO:85.

Amplification oligomer pairs include, primer pairs, a primer member and a promoter-based amplification oligomer member, and tagged amplification oligomers.

In one embodiment, amplification oligomer combinations comprise a primer oligomer member and a promoter-based oligomer member. Preferably, a promoter-based amplification oligomer is a promoter primer comprising a 5′ RNA polymerase promoter sequence and a 3′ target binding sequence. RNA polymerase promoter sequences are known in the art to include, but not be limited to, sp6 RNA polymerase promoter sequences, T3 RNA polymerase promoter sequences and T7 RNA polymerase promoter sequences. In the preferred embodiments, a promoter primer comprises a 5′ T7 RNA polymerase promoter sequence and a 3′ target binding sequence. Most preferably, the 5′ T7 RNA polymerase promoter sequence is SEQ ID NO:49.

In one embodiment, the 3′ target binding sequence of a promoter-based amplification oligomer is from about 10 to about 70 nucleobases in length and comprises, consists or consists essentially of a nucleic acid sequence that is configured to specifically hybridize to a region within a target sequence of an MLV nucleic acid, wherein said region is from residue 2924 to residue 2971 or from residue 7756 to residue 7804 of SEQ ID NO:85 (GenBank Accession Number EF185282.1 GI:121104176 entered at NCBI on Jan. 10, 2007). Exemplary promoter based amplification oligomers comprise, consist or consist essentially of a target binding sequence as set forth in SEQ ID NOS: 125, 127, 130, 131, 157, or 159. Exemplary promoter based amplification oligomer target hybridizing sequences are those that are substantially identical to one of SEQ ID NOS: 122 through 133. Exemplary promoter based amplification oligomers are those that are substantially identical to one of SEQ ID NOS: 110 through 121. Moreover, it is recognized that insert sequences can be included with any of the promoter-based oligomer members described herein.

In one embodiment, the amplification oligomer combination comprises at least one primer amplification oligomer member. Primer amplification oligomers have a length that is from about 10 nucleobases to about 50 nucleobases, and have a nucleotide composition configured to specifically hybridize with MLV to generate a detectable amplification product when used in an amplification reaction of the current invention. Primer target binding sequences include those that are configured to specifically hybridize all or a portion of a region of a target sequence of a MLV, wherein said region corresponds to from residue 2800 to 2862 or from residue 7676 to residue 7713 of SEQ ID NO:85. Exemplary primers comprise, consist or consist essentially of a target binding sequence that contains SEQ ID NOS: 99, 102, 103, 109, or 158. Exemplary primers comprise target hybridizing sequences that are substantially identical to SEQ ID NOS: 97 through 109. Moreover, it is recognized that 5′ tag sequences can be included with any of the primer oligomer members of the current invention. 5″ tag sequences are sequences that are configured to not hybridize with a target nucleic acid. Tag sequences are often incorporated into amplification products to serve a function, such as primer binding sites for subsequent rounds of amplification, or other function.

Amplifying the target nucleic acid by TMA produces many strands of nucleic acid amplification product from a single copy of target nucleic acid, thus permitting detection of the target using detecting probes that hybridize to the sequences of the amplification product. Generally, the reaction mixture includes the target nucleic acid and at least two amplification oligomers comprising at least one primer, at least one promoter primer, reverse transcriptase and RNA polymerase activities, nucleic acid synthesis substrates (deoxyribonucleoside triphosphates and ribonucleoside triphosphates) and appropriate salts and buffers in solution to produce multiple RNA transcripts from a nucleic acid template. Briefly, a promoter-primer hybridizes specifically to a portion of the target sequence. Reverse transcriptase that includes RNase H activity creates a first strand cDNA by 3′ extension of the promoter-primer. The cDNA is hybridized with a primer downstream from the promoter primer and a new DNA strand is synthesized from the 3′ end of the primer using the reverse transcriptase to create a dsDNA having a functional promoter sequence at one end. RNA polymerase binds to dsDNA at the promoter sequence and transcribes multiple transcripts or amplicons. These amplicons are further used in the amplification process, serving as a template for a new round of replication, to ultimately generate large amounts of single-stranded amplified nucleic acid from the initial target sequence (e.g., 100 to 3,000 copies of RNA synthesized from a single template). The process uses substantially constant reaction conditions (i.e., substantially isothermal).

TMA reactions are also performed using a combination of amplification oligomers, wherein said combination comprises at least two promoter primer oligomer members and at least two primer members. One combination of amplification oligomers for performing a multiplex amplification reaction comprises an amplification oligomer pair targeting the polymerase gene (POL) of MLV and an amplification oligomer pair targeting the LTR gene of MLV. One combination of amplification oligomers for multiplex TMA includes a first pair of amplification oligomers configured to generate from SEQ ID NO:85 an amplification product containing a nucleotide sequence corresponding to from about residue 2862 to about residue 2971, and a second pair of amplification oligomers configured to generate from SEQ ID NO:85 an amplification product containing a nucleotide sequence corresponding to from about residue 7713 to about 7756. Amplification oligomers are preferably of a length less than 200 nucleobases.

In one embodiment, at least two amplification oligomers amplify at least a portion of the POL gene of MLV target nucleic acid. According to this embodiment of the invention, the first amplification oligomer comprises, consists or consists essentially of a target hybridizing sequence 10 to 50 nucleotides in length and is configured to target a sequence corresponding to nucleotides 2924 to 2971 of SEQ ID No. 85. The first amplification oligomer may comprise, consist or consist essentially of a target hybridizing sequence configured to target a sequence in a region corresponding to nucleotides 2926 to 2944 or nucleotides 2934 to 2951 or nucleotides 2954 to 2971 of SEQ ID No. 85.

Suitably, the first amplification oligomer comprises, consists or consists essentially of a target hybridizing sequence configured to target a sequence in a region corresponding to nucleotides 2924 to 2942; or 2926 to 2946; or nucleotides 2933 to 2951; or nucleotides 2934 to 2951; or nucleotides 2953 to 2971; or nucleotides 2954 to 2971 of SEQ ID No. 85.

Suitably, the first amplification oligomer comprises, consists or consists essentially of the sequence set forth in any of SEQ ID Nos. 110 to 115 or 122 to 127.

Suitably, the second of said amplification oligomers comprises a target hybridizing sequence about 10 to 50 nucleotides in length and is configured to target a sequence corresponding to nucleotides 2800 to 2862 of SEQ ID No. 85. The second amplification oligomer may comprise, consist or consist essentially of a target hybridizing sequence configured to target a sequence in a region corresponding to nucleotides 2801 to 2817 or nucleotides 2832 to 2853 or nucleotides 2842 to 2859 of SEQ ID No. 85.

Suitably, said second amplification oligomer comprises, consists or consists essentially of a target hybridizing sequence configured to target a sequence in a region corresponding to nucleotides 2800 to 2817; or nucleotides 2801 to 2817; or nucleotides 2830 to 2853; or nucleotides 2832 to 2853; or nucleotides 2842 to 2859; or nucleotides 2842 to 2861; or nucleotides 2842 to 2862 of SEQ ID No. 85.

Suitably, the second amplification oligomer comprises, consists or consists essentially of the sequence set forth in any of SEQ ID Nos. 97 to 104.

In another embodiment, at least two amplification oligomers amplify at least a portion of the LTR gene of MLV target nucleic acid. According to this embodiment of the invention, a first of said amplification oligomers comprises a target hybridizing sequence 10 to 50 nucleotides in length and is configured to target a sequence corresponding to nucleotides 7756 to 7804 of SEQ ID No. 85. The first amplification oligomer may comprise, consist or consist essentially of a target hybridizing sequence configured to target a sequence in a region corresponding to nucleotides 7758 to 7778 or nucleotides 7769 to 7784 or nucleotides 7787 to 7801.

Suitably, said first amplification oligomer comprises, consists or consists essentially of a target hybridizing sequence configured to target a sequence in a region corresponding to nucleotides 7756 to 7778; or nucleotides 7757 to 7778; or nucleotides 7758 to 7778; or nucleotides 7769 to 7784; or nucleotides 7786 to 7801; or nucleotides 7787 to 7804 of SEQ ID No. 85.

Suitably, said first amplification oligomer comprises, consists or consists essentially of the sequence set forth in any of SEQ ID Nos. 116 to 121 or 128 to 133.

Suitably, the second of said amplification oligomers comprises a target hybridizing sequence 10 to 50 nucleotides in length and is configured to target a sequence corresponding to nucleotides 7676 to 7713 of SEQ ID No. 85. The second amplification oligomer may comprise, consist or consist essentially of a target hybridizing sequence configured to target a sequence in a region corresponding to nucleotides 7678 to 7697 or nucleotides 7689 to 7710 with an extra “T” inserted between bases 7691 and 7692.

Suitably, the second amplification oligomer comprises, consists or consists essentially of a target hybridizing sequence configured to target a sequence in a region corresponding to nucleotides 7676 to 7697; or nucleotides 7678 to 7699; or nucleotides 7678 to 7700; or nucleotides 7689 to 7713; or nucleotides 7689 to 7710 with an extra “T” inserted between bases 7691 and 7692.

Suitably, the second amplification oligomer comprises, consists or consists essentially of the sequence set forth in any of SEQ ID Nos. 105 to 109.

A further aspect relates to the amplification of more than one region of MLV nucleic acid using combinations of amplification oligomers. In one embodiment, a multiplex amplification method is provided in which amplification oligomers configured to amplify nucleic acid using at least two pairs of amplification oligomers. Thus, for example, amplification oligomers configured to amplify at least a portion of the Pol gene of MLV and amplification oligomers configured to amplify at least a portion of the LTR of MLV are used in combination.

Accordingly, there is provided in a further embodiment, a method for the multiplex amplification and identification of an MLV from a sample comprising the steps of: (a) contacting a sample suspected of containing MLV with at least two pairs of amplification oligomers as described herein for generating separate amplicons from an MLV target nucleic acid, wherein each pair of amplification oligomers is from 10 to about 50 nucleotides in length and wherein a first amplification oligomer pair is configured to specifically hybridize to regions within a target sequence of MLV comprising, consisting or consisting essentially of from residue 2800 to residue 2862 and from residue 2924 to residue 2971 of SEQ ID NO:85, and wherein a second amplification oligomer pair is configured to specifically hybridize to regions within a target sequence of MLV comprising, consisting or consisting essentially of from residue 7676 to residue 7713 and from residue 7756 to residue 7804 of SEQ ID NO:85; (b) providing conditions sufficient for generating amplicons from the MLV target nucleic acid present in said sample using said amplification oligomers from step (a); and (c) providing conditions for detecting said amplicon and determining whether said sample contained MLV target nucleic acid.

Combinations of oligomers and probes that can be used for the detection of MLV are also disclosed.

By way of example, combinations of oligomers and probes that can be used for the detection of the Pol gene of MLV are disclosed.

(i) One preferred combination of amplification oligomers comprises the target-specific sequence of SEQ ID No. 122, which is contained in the promoter primer of SEQ ID No. 110, in combination with any one or more of SEQ ID Nos. 97, 99, and 103.

(ii) Another preferred combination of amplification oligomers comprises the target-specific sequence of SEQ ID No. 123, which is contained in the promoter primer of SEQ ID No. 111, in combination with any one or more of SEQ ID Nos. 97 and 99.

(iii) Another preferred combination of amplification oligomers comprises the target-specific sequence of Seq ID No. 124, which is contained in the promoter primer of SEQ ID No. 112, in combination with Seq ID Nos. 99 and 103.

(iv) Another preferred combination of amplification oligomers comprises the target-specific sequences of Seq ID No. 125, which is contained in the promoter primer of Seq ID No. 113, in combination with any one or more of SEQ ID No. 98.

(v) Another preferred combination of amplification oligomers comprises the target-specific sequence of SEQ ID No. 126, which is contained in the promoter primer of SEQ ID No. 114, in combination with any one or more of SEQ ID Nos. 97, 98, 99, and 104.

(vi) Another preferred combination of amplification oligomers comprises the target-specific sequences of SEQ ID No. 127, which is contained in the promoter primer of SEQ ID No. 115, in combination with any one or more of SEQ ID Nos. 97 and 98.

(vii) A specific combination of amplification oligomers comprises the target-specific sequences of SEQ ID No. 122, which is contained in the promoter primer of SEQ ID No. 110, in combination with SEQ ID Nos. 97.

(viii) Another specific combination of amplification oligomers comprises the target-specific sequences of SEQ ID No. 122, which is contained in the promoter primer of SEQ ID No. 110, in combination with SEQ ID No. 103.

(ix) Another specific combination of amplification oligomers comprises the target-specific sequences of SEQ ID No. 127, which is contained in the promoter primer of SEQ ID No. 115, in combination with SEQ ID No. 97.

(x) Another specific combination of amplification oligomers comprises the target-specific sequences of SEQ ID No. 127, which is contained in the promoter primer of SEQ ID No. 115, in combination with SEQ ID No. 103.

(xi) Another specific combination of amplification oligomers comprises the combination of target-specific sequences of SEQ ID No. 122, which is contained in the promoter primer of SEQ ID No. 110, SEQ ID No. 127, which is contained in the promoter primer of SEQ ID No. 115, SEQ ID No. 97, and SEQ ID No. 103.

In one embodiment, one or more primer combinations (i) to (xi) may be used in combination with a probe comprising, consisting of consisting essentially of the sequences set forth in SEQ ID Nos. 148, 149, and 150.

In one embodiment, one or more primer combinations (i) to (xi) may be used in combination with a target capture oligonucleotide comprising, consisting of consisting essentially of the sequence set forth in SEQ ID Nos. 134, 135, 136, 141, 142, and 143 and optionally together with a probe comprising, consisting of consisting essentially of the sequences set forth in SEQ ID No. 148, 149, and 150.

Combinations of oligomers and probes that can be used for the amplification and detection of LTR of MLV are also disclosed.

(i) One preferred combination of amplification oligomers comprises the target-specific sequence of SEQ ID No. 128, which is contained in the promoter primer of SEQ ID No. 116, in combination with any one or more of SEQ ID Nos. 105, 106, 107, 108, and 109.

(ii) Another preferred combination of amplification oligomers comprises the target-specific sequence of SEQ ID No. 129, which is contained in the promoter primer of SEQ ID No. 117, in combination with any one or more of SEQ ID Nos. 105, 106, 107, 108, and 109.

(iii) Another preferred combination of amplification oligomers comprises the target-specific sequence of SEQ ID No. 130, which is contained in the promoter primer of SEQ ID No. 118, in combination with SEQ ID No. 105, 106, 107, 108, and 109.

(iv) Another preferred combination of amplification oligomers comprises the target-specific sequences of SEQ ID No. 131, which is contained in the promoter primer of SEQ ID No. 119, in combination with any one or more of SEQ ID Nos. 105, 106, and 108.

(v) Another preferred combination of amplification oligomers comprises the target-specific sequence of SEQ ID No. 132, which is contained in the promoter primer of SEQ ID No. 120, in combination with any one or more of SEQ ID Nos. 107, 108, and 109.

(vi) Another preferred combination of amplification oligomers comprises the target-specific sequences of SEQ ID No. 133, which is contained in the promoter primer of SEQ ID No. 121, in combination with any one or more of SEQ ID Nos. 108 and 109.

(vii) A specific combination of amplification oligomers comprises the target-specific sequences of SEQ ID No. 128, which is contained in the promoter primer of SEQ ID No. 116, in combination with any one or more of SEQ ID No. 108.

(viii) Another specific combination of amplification oligomers comprises the target-specific sequences of SEQ ID No. 130, which is contained in the promoter primer of SEQ ID No. 118, in combination with any one or more of SEQ ID No. 108.

In one embodiment, one or more primer combinations (i) to (viii) may be used in combination with a probe comprising, consisting of consisting essentially of the sequence set forth in SEQ ID Nos. 151, 152, 153, 154, 155, and 156.

In one embodiment, one or more primer combinations (i) to (viii) may be used in combination with a target capture oligonucleotide comprising, consisting of consisting essentially of the sequence set forth in SEQ ID Nos. 137, 138, 139, 140, 144, 145, 146, and 147 and optionally together with a probe comprising, consisting of consisting essentially of the sequence set forth in SEQ ID Nos. 151, 152, 153, 154, 155, and 156.

Combinations of each of the oligomers (i) to (xi) that can be used for the amplification of Pol of MLV may be used in combination with each of the oligomers (i) to (viii) that can be used for the amplification of LTR of MLV.

Thus, for example, Pol oligomer (i) can be used together with any one of LTR oligomers (i) to (viii); Pol oligomers (ii) can be used together with any one of LTR oligomers (i) to (viii); Pol oligomers (iii) can be used together with any one of LTR oligomers (i) to (viii); Pol oligomer (iv) can be used together with any one of LTR oligomers (i) to (viii); Pol oligomer (v) can be used together with any one of LTR oligomers (i) to (viii); Pol oligomer (vi) can be used together with any one of LTR oligomers (i) to (viii); Pol oligomer (vii) can be used together with any one of LTR oligomers (i) to (viii); Pol oligomer (viii) can be used together with any one of LTR oligomers (i) to (viii); Pol oligomer (xi) can be used together with any one of LTR oligomers (i) to (viii); Pol oligomer (x) can be used together with any one of LTR oligomers (i) to (viii); Pol oligomer (xi) can be used together with any one of LTR oligomers (i) to (viii).

By way of further example, LTR oligomer (i) can be used together with any one of Pol oligomers (i) to (xi); LTR oligomer (ii) can be used together with any one of Pol oligomers (i) to (xi); LTR oligomers (iii) can be used together with any one of Pol oligomers (i) to (xi); LTR oligomer (iv) can be used together with any one of Pol oligomers (i) to (xi); LTR oligomer (v) can be used together with any one of Pol oligomers (i) to (xi); LTR oligomer (vi) can be used together with any one of Pol oligomers (i) to (xi); LTR oligomer (vii) can be used together with any one of Pol oligomers (i) to (xi); LTR oligomer (viii) can be used together with any one of Pol oligomers (i) to (xi).

The combinations of amplification oligomers described herein above may be used together with one or more of the detection probes described herein. Accordingly, further combinations of oligonucleotides according to the present invention include each of the combinations set forth above together with one or more detection probes as described herein. The combinations of amplification oligomers or the combinations of amplification oligomers and detection probes described above may be also be used in combination with one or more target capture oligomers as described herein.

### Amplification of the MLV Target Nucleic Acid

Described herein are compositions, reaction mixtures, kits and methods useful in the identification of MLV from a sample. MLV target nucleic acids are separated from other sample components using target capture compositions, mixtures and methods described herein. Using the target capture compositions, mixtures and methods described herein, any target nucleic acid present in a sample is removed and then can be further assayed to determine its presence, or, optionally, specific identification. The further assay methods can be any known in the art that provide a desired determination of the presence or identification of a target nucleic acid. Captured target nucleic acid can be sequenced, amplified, analyzed by mass spectrometry, or otherwise assayed.

RNA target nucleic acids are typically converted to DNA and are then amplified as double stranded DNA target nucleic acids. This step is referred to as reverse transcription, and it uses an RNA-dependent DNA polymerase or reverse transcriptase (“RT”), which is an enzyme that synthesizes a complementary DNA copy from an RNA template. All known reverse transcriptases also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

Amplification assays include PCR, wherein the captured target nucleic acid is contacted with a primer pair and a polymerase. (U.S. Pat. Nos. 4,683,202; 4,683,195; 4,800,159; and 4,965,188). In its simplest form, PCR is an in vitro method for the enzymatic synthesis of specific nucleic acid sequences, using two oligonucleotide primers that hybridize to opposite strands and flank the region of interest in the target nucleic acid. A repetitive series of reaction steps involving template denaturation, primer annealing, and the' extension of the annealed primers by polymerase results in the exponential accumulation of a specific fragment whose termini are defined by the 5′ ends of the primers. PCR is capable of producing a selective enrichment of a specific DNA sequence by a factor of 10.sup.9. The PCR method is also described in Saiki et al., 1985, Science 230:1350. A variation on the PCR reaction or RT-PCR reaction is real time PCR. A common real-time PCR method is taqman PCR (U.S. Pat. Nos. 5,210,015 and 5,538,848), though other methods are well known in the art. Compositions, reaction mixtures and methods described herein are useful for PCR amplification of MLV target nucleic acids. Preferably, the PCR reaction is an RT-PCR amplification reaction.

Amplification assays also include isothermal amplification assays wherein the target nucleic acid is amplified under a substantially constant temperature. This is in contract to the series of temperature cycles used in PCR. Transcription Mediated Amplification (TMA) is an isothermal nucleic acid amplification that uses an RNA polymerase to produce multiple RNA transcripts from a target nucleic acid. TMA generally employs RNA polymerase and DNA polymerase activities, deoxyribonucleoside triphosphates, ribonucleoside triphosphates, and a promoter-based amplification oligomer, and optionally may include one or more other oligonucleotides, including “helper” or “blocker” oligomers. Variations of transcription-associated amplification are well known in the art and described in detail elsewhere (see U.S. Pat. Nos. 5,399,491 and 5,554,516 to Kacian et al., 5,437,990 to Burg et al., 5,130,238 to Malek et al., 4,868,105 and 5,124,246 to Urdea et al., PCT No. WO 93/22461 by Kacian et al., PCT Nos. WO 88/01302 and WO 88/10315 by Gingeras et al., PCT No. WO 94/03472 by McDonough et al., and PCT No. WO 95/03430 by Ryder et al.). The procedures of U.S. Pat. Nos. 5,399,491 and 5,554,516 are preferred amplification embodiments.

Following sample preparation, amplification of an MLV target was achieved by using amplification oligomers that define the 5′ and 3′ ends of the target sequence amplified by in vitro enzyme-mediated nucleic acid synthesis to generate an amplicon. TMA methods, substantially as described in U.S. Pat. Nos. 5,399,491 and 5,554,516, produce a large number of amplification products (RNA transcripts) that can be detected. Preferred embodiments of the method used mixtures of amplification oligomers in which at least one promoter primer is combined with at least one primer.

### Sample Preparation

Preparation of samples for amplification and detection of MLV sequences may include methods of separating and/or concentrating viruses contained in a sample from other sample components. Sample preparation may include routine methods of disrupting samples or lysing samples to release intracellular contents, including MLV nucleic acids or genetic sequences encoding the MLV or a fragment thereof. Sample preparation before amplification may include an optional step of target capture to specifically or non-specifically separate the target nucleic acids from other sample components. Nonspecific target capture methods may involve selective precipitation of nucleic acids from a substantially aqueous mixture, adherence of nucleic acids to a support that is washed to remove other sample components, other methods of physically separating nucleic acids from a mixture that contains MLV nucleic acid and other sample components.

In one embodiment, MLV target nucleic acids are selectively separated from other sample components by specifically hybridizing the MLV target nucleic acid to a capture oligomer specific for MLV to form a target sequence:capture probe complex. The complex is separated from sample components by binding the target:capture probe complex to an immobilized probe, and separating the target:capture probe:immobilized probe complex from the sample, as previously described (U.S. Pat. Nos. 6,110,678; 6,280,952; and 6,534,273). Target capture may occur in a solution phase mixture that contains one or more capture oligonucleotides that hybridize specifically to target nucleic acids under hybridizing conditions, usually at a temperature higher than the Tm of the tail sequence:immobilized probe sequence duplex. The target:capture probe complex is captured by adjusting the hybridization conditions so that the capture probe tail hybridizes to the immobilized probe, and the entire complex on the support is then separated from other sample components. The support with the attached immobilized probe:capture probe:target sequence may be washed one or more times to further remove other sample components. Other embodiments link the immobilized probe to a particulate support, such as a paramagnetic bead, so that particles with the attached target:capture probe:immobilized probe complex may be suspended in a washing solution and retrieved from the washing solution, by using magnetic attraction. To limit the number of handling steps, the target nucleic acid may be amplified by simply mixing the target sequence in the complex on the support with amplification oligonucleotides and proceeding with amplification steps.

Capture probes including a dT₃A₃₀ tail portion are suitable for hybridization to a complementary immobilized sequence, whereas capture probes without this tail portion can be used in conjunction with another ligand that is a member of a binding pair (eg., biotinylated DNA to bind to immobilized avidin or streptavidin). The complex of the capture probe, its target MLV nucleic acid, and an immobilized binding partner or probe facilitate separation of the MLV nucleic acid from other sample components, and optional washing steps may be used to further purify the captured viral nucleic acid.

Preferred embodiments of target capture oligomers include a target-specific sequence that binds specifically to the MLV target nucleic acid and a covalently linked “tail” sequence (eg. T₀₋₄A₁₀₋₃₆) used in capturing the hybridization complex containing the target nucleic acid to an immobilized sequence on a solid support. Capture oligomers may include at least one 2′ methoxy linkage. Embodiments of capture oligomers may include the target-specific sequence that binds to MLV nucleic acid attached to another binding moiety, e.g., a biotinylated sequence that binds specifically to immobilized avidin or streptavidin. The tail sequence or binding moiety binds to an immobilized probe (eg., complementary sequence or avidin) to capture the hybridized target and separate it from other sample components by separating the solid support from the mixture.

Exampleary target capture oligonucleotides for use in capturing MLV nucleic acid include SEQ ID Nos. 134 to 147.

### Nucleic Acid Detection

Detection of the nucleic acids may be accomplished by a variety of methods. Detection methods may use nucleic acid probes comprising a target hybridizing sequence that is complementary to a portion of the amplified product and detecting the presence of the probe:product complex, or by using a complex of probes that may amplify the detectable signal associated with the amplified products (e.g., U.S. Pat. Nos. 5,424,413; 5,451,503; and 5,849,481). Directly or indirectly labeled probes that specifically associate with the amplified product provide a detectable signal that indicates the presence of the target nucleic acid in the sample. For example, if the target nucleic acid is MLV RNA, the amplified product will contain a sequence in or complementary to a MLV target sequence. A probe is configured to bind directly or indirectly to a portion of the amplification product to indicate the presence of MLV in the tested sample,

Probes that hybridize to the amplified sequences include hairpin oligonucleotides such as Molecular Torches and linear oligonucleotides that substantially do not form conformations held by intramolecular bonds. Preferably, said probes may include labels. Linear probe embodiments may include a chemiluminescent compound as the label, e.g. a chemiluminescent AE compound attached to the probe sequence via a linker (substantially as described in U.S. Pat. Nos. 5,585,481 and 5,639,604, particularly at column 10, line 6 to column 11, line 3, and in Example 8 therein). Examples of labeling positions are a central region of the probe oligomer and near a region of A:T base pairing, at a 3′ or 5′ terminus of the oligomer, and at or near a mismatch site with a known sequence that is not the desired target sequence. Hairpin or linear probes may be labeled with any of a variety of different types of interacting labels, where one interacting member is usually attached to the 5′ end of the probe and the other interacting member is attached to the 3′ end of the probe. Dye labeled probes, including dual labeled probes, single labeled probes, AE labeled probes and the like, are generally known. Dual labeled probes can be labeled at one end with a fluorescent label (“F”) that absorbs light of a particular wavelength or range and emits light another emission wavelength or range and at the other end with a quencher (“Q”) that dampens, partially or completely, signal emitted from the excited F when Q is in proximity with the fluorophore. Such a probe may be referred to as labeled with a fluorescent/quencher (F/Q) pair. One embodiment of a hairpin probe is a “molecular torch” that detects an amplified product to indicate whether a target sequence is present in the sample after the amplification step. A molecular torch probe comprises a target binding domain and a closing domain, as is described above. These domains allow the molecular torch to exist in open and closed conformations, depending on whether the torch is bound to a target. (See also, U.S. Pat. Nos. 6,849,412; 6,835,542; 6,534,274; and 6,361,945). Another hairpin probe embodiment is a “molecular beacon” which is generally described in Tyagi et al., 1998, Nature Biotechnol. 16:49-53, and in U.S. Pat. Nos. 5,118,801; and 5,312,728. Methods for using such hairpin probes to detect the presence of a target sequence are well known in the art.

Exemplary probes for use in the detection of MLV nucleic acid include SEQ ID Nos. 148 to 156.

One method for detecting MLV sequences may use a transcription associated amplification together with a molecular torch. The molecular torch is added before or during amplification, allowing detection to be carried out without the addition of other reagents. For example, a molecular torch may be designed so that the Tm of the hybridized target binding region and closing region complex is higher than the amplification reaction temperature, thusly designed to prevent the probe from prematurely binding to amplified target sequences. After an interval of amplification, the mixture is heated to open the torch regions and allow the target binding regions to hybridize to a portion of the amplification products. The solution is then cooled to close any probes not bound to amplified products by allowing the probe target binding and closing regions to hybridize, which effectively closes the label/quencher pair. Detection is then performed to generate and detect signals from only the probes that are hybridized to the amplified target sequences. For example, the mixture containing the F/Q labeled hairpin probe is irradiated with the appropriate excitation light and the emission signal is measured. In other embodiments, the hairpin detection probe is designed so that the amplified products hybridize to the target binding region of the probe during amplification, resulting in changing the hairpin to its open conformation during amplification, and the amplification reaction mixture is irradiated at intervals to detect the emitted signal from the open probes in real time during amplification.

The MLV assays may use amplification systems that are detected during the amplification process (e.g., real time detection) by including probes that emit distinguishable signals—such as fluorescent signals—when the probe is bound to the intended target sequence made during the amplification process. Thus, according to one embodiment, different probes emit distinguishable different signals. Probes for real time detection include those referred to as “molecular beacon” or “molecular switch” probes (e.g., U.S. Pat. Nos. 5,118,801 and 5,312,728, Lizardi et al., U.S. Pat. Nos. 5,925,517 and 6,150,097, Tyagi et al., Giesendorf et al., 1998, Clin. Chem. 44(3):482-6) and “molecular torch” probes (e.g., U.S. Pat. Nos. 6,835,542 and 6,849,412, Becker et al.). Generally, such probes include a reporter dye attached to one end of the probe oligomer (e.g., FAM™, TET™, JOE™, VIC™) and a quencher compound (e.g., TAMRA™ or non-fluorescent quencher) attached to the other end of the probe oligomer, and signal production depends on whether the two ends with their attached compounds are in close proximity or separated.

### Exemplary Method for the Amplification and Detection of MLV Nucleic Acid

In general, methods used to demonstrate amplification and detection of MLV nucleic acid by using the compositions described herein involve the following steps. RNA is separated from other sample components by using a method that attaches the target nucleic acid to a solid support that is separated from other sample components. In preferred embodiments, viral RNA is separated from other sample components by using a target-capture system that includes a target-specific capture probe for the MLV viral analyte (e.g., using methods steps described in U.S. Pat. Nos. 6,110,678, 6,280,952 and 6,534,273), or a non-specific method for separation of nucleic acids was used (U.S. Pat. No. 5,234,809). Non-specific separation of viral RNA from other sample components is performed by adhering nucleic acids reversibly to a solid support, followed by washing and elution of the adhered nucleic acids into a substantially aqueous solution (e.g., using a QIAAMP™ Viral RNA Mini kit, Qiagen Inc.). Isolated MLV nucleic acid is amplified for specific target sequences contained in the genome by using TMA amplification, and the amplification products are detected after completion of the amplification reaction. Signal can be detected by using a system that incubates the reactions and detects fluorescence at different wavelengths (eg., using a DNA Engine OPTICON™ 2 system or CHROMO4™ Real-Time PCR Detector, Bio-Rad Laboratories, Inc., Hercules, Calif.).

Real-time TMA-based assays may also be used. These assays are typically performed in reaction mixture that contains the analyte nucleic acid, amplification reagent (eg. APTIMA™ reagent, Gen-Probe Incorporated, San Diego, Calif.), a T7 promoter primer (eg. about 5 pmol/reaction), a second primer without a promoter (eg. about 5 pmol/reaction), and a detection probe (eg. about 0.2-0.3 pmol/reaction) for amplicon detection, in a 40.micro.l reaction (in a well of a standard 96-well plate, covered with a layer of inert oil or sealing device to prevent evaporation). The mixture of target nucleic acid, primers, and probe may be incubated at about 60.deg. C. for about 10 min, cooled to about 42.deg. C. for about 5 min, and then enzyme reagent containing RT and T7 RNA polymerase is added, the mixture is mixed (e.g., 30 sec vortex) and then incubated at about 42.deg. C. for about 75-100 min for isothermal amplification during which detection of fluorescence is performed either during the reaction (eg. every 3 seconds) or at the end of the reaction. Amplification and detection steps may be performed using an incubation and open channel fluorimeter (eg. CHROMO4™, Bio-Rad Laboratories, Inc.) for real-time two-color fluorescence detection. The assays may include an IC, as described above, i.e., a reaction mixture included primers and probe for the target MLV nucleic acid and IC-specific primers and probe, each probe labeled with a separately detectable 5′ fluorophore. Real-time fluorescence signals are analyzed and a detection signal (time of emergence) is calculated. Time of emergence is calculated, e.g., by using a method that analyzes the detected signals (relative fluorescence units or RFU) relative to the signal detection times (RFU(t) data points) to determine a time of emergence (“T-time”), which is the time at which a RFU(t) data point reaches a predefined threshold value (described in detail in U.S. application 60/659,874, Scalese et al., filed Mar. 10, 2005; and US published application US2007-0243600). Briefly, RFU(t) data is treated to subtract background signal (“noise” level) and curves (RFU vs time) is normalized to optimize curve fit for data between predetermined minimum and maximum points. In general, samples that contain a higher analyte concentration result in a steeper curve slope and an earlier time of emergence. Average times of emergence are compared to determine the relative efficiencies of the different assay conditions, e.g., to compare for a single known amount of analyte, the time of emergence detected by using a PCR-based assay compared to using a TMA-based assay.

### Kits

The oligomers for use in the methods described herein are suited for preparation of kits. Such a kit may comprise containers, each with one or more of the various oligomers optionally together with one or more of the reagents (eg. enzymes) required to perform the methods described herein. The components of the kit may be supplied in concentrated form. A set of instructions for using the components of the kit will also typically be included. Where the kit comprises combinations of oligomers then the individual oligomers may be provided in individual form, with appropriate instructions for mixing same, or combinations thereof that are ready mixed.

Correlation of Detection of a Target Sequence with Diagnosis

The detection of amplified target sequences characteristic of MLV in a biological sample from an individual is indicative of the presence of MLV.

### EXAMPLES

### Example 1

### Reagents for TMA-Based Assays

Unless otherwise specified, reagents commonly used in the TMA-based assays described herein include the following. Sample Transport Reagent: 110 mM lithium lauryl sulfate (LLS), 15 mM NaH₂PO₄, 15 mM Na₂HPO₄, 1 mM EDTA, 1 mM EGTA, pH 6.7. Lysis Buffer: 790 mM HEPES, 230 mM succinic acid, 10% (w/v) LLS, and 680 mM LiOH monohydrate. Target Capture Reagent (TCR): lysis buffer containing 250.micro.g/ml of paramagnetic particles (0.7-1.05 micron particles, Sera-Mag™ MG-CM) with (dT)₁₄ oligomers covalently bound thereto and one or more target capture oligomers. Wash Solution: 10 mM HEPES, 150 mM NaCl, 6.5 mM NaOH, 1 mM EDTA, 0.3% (v/v) ethanol, 0.02% (w/v) methylparaben, 0.01% (w/v) propylparaben, and 0.1% (w/v) sodium lauryl sulfate, pH 7.5. Amplification Reagent: typical 100 μl amplification reactions use 75 μl of an amplification reagent mixture containing 10-12 mM Tris Base, 13-15 mM Tris-HCl, 22-25.5 mM MgCl2, 22-25.5 mM KCl, 2-4.5% glycerol, 0.03 to 0.09 mM Zn-acetate (dihydrate), 0.5-1.0 mM each of dATP, dCTP, dGTP, and dTTP, 5 to 10 mM each of ATP, CTP, GTP, and UTP, pH 7) and 25 μl of an enzyme reagent mixture (600 to 900 U of T7 RNA polymerase, 1000-1400 U of reverse transcriptase from Moloney Murine Leukemia Virus (MMLV-RT), 15 to 18 mM HEPES (free acid, dihydrate), 50-100 mM N-acety-L-cysteine, EDTA, Na-azide, 20 to 23 mM Tris base, 50 to 60 mM KCl, 18-20% (v/v) anhydrous glycerol, 10-11% (v/v) TRITON® X-102, and 150 to 180 mM trehalose (dihydrate), pH 7), preferably mixed with the captured target nucleic acid retained on the solid particles. Probe Reagent for AE-labeled probes: typically includes 100 mM succinate, 2% (w/v) LLS, 230 mM LiOH (monohydrate), 15 mM 2,2′-dithiodipyridine (ALDRITHIOL-2), 1.2 M LiCl, 20 mM EDTA, 20 mM EGTA, 3% (v/v) absolute ethanol, brought to about pH 4.7 with LiOH, and the selection reagent used for hydrolyzing the label on unbound probe included 600 mM boric acid, 182 mM NaOH, 1% (v/v) TRITON® X-100. Signals are detected as relative light units (RLU) using a luminometer (e.g., LEADER™ 450HC+, Gen-Probe Incorporated, San Diego, Calif.). Detection Reagents for AE labels are Detect Reagent I: 1 mM nitric acid and 32 mM H₂O₂, and Detect Reagent II: 1.5 M NaOH (see U.S. Pat. Nos. 5,283,174, 5,656,744, and 5,658,737).

### Example 2

### Testing POL Specific Primers Designed to Amplify MLV on In Vitro Transcribed RNA

The purpose of this experiment was to test the POL specific primers for their ability to detect and amplify in vitro transcribed RNA (SEQ ID NO:96). Primers consisting of SEQ ID Nos. 97 to 104 (Non-T7) and SEQ ID Nos. 110 to 115 (T7) were tested.

The amplification mixture used contained 5 pmol of T7 primer and 5 pmol of nonT7 primer per reaction. 0 or 10 copies of in vitro transcribed RNA (SEQ ID NO:96) was used per reaction. 200 microlitres of oil was added to each tube and vortexed, followed by incubation at 62.deg. C. for 10 minutes followed by 41.5.deg. C. for 20 minutes. 25 microlitres of enzyme was added to each tube, hand shaken and the incubation continued at 41.5.deg. C. for 50 minutes. 100 microlitres of probe reagent (AE labeled SEQ ID No. 148 at 2.5E6 RLU/100 ul of hybridizing reagent) was added, vortexted and incubated at 62.deg. C. for 20 minutes. 250 microlitres of selection reagent was then added and the tube vortexed, followed by incubation at 62.deg. C. for 10 minutes. Detect reagent I and II were added immediately before measuring the relative light units (RLU).

The results are shown below in Table 1, representing the RLU obtained for each reaction Positive results are indicated in bold.

TABLE 1
Amplification Oligomer Combinations

| SEQ | Copies/ | | | | | | |
|---|---|---|---|---|---|---|---|
| ID No. | RXN | 110 | 111 | 112 | 113 | 114 | 115 |
| 97 | 0 | 76 | 709 | 655 | 1,813 | 678 | 638 |
| | 10 | 5,680 | 696 | 750 | 955 | 18,969 | 2,170 |
| | 10 | 542,058 | 19,983 | 725 | 998 | 3,157 | 3,526 |
| 98 | 0 | 699 | 681 | 733 | 1,122 | 591 | 719 |
| | 10 | 612 | 646 | 722 | 1,115 | 875 | 4,596 |
| | 10 | 736 | 600 | 879 | 12,738 | 3,925 | 791 |
| 99 | 0 | 856 | 6,102 | 730 | 928 | 687 | 948 |
| | 10 | 267,747 | 16,233 | 876 | 918 | 957 | 832 |
| | 10 | 50,631 | 787 | 52,490 | 814 | 745 | 768 |
| 100 | 0 | 760 | 699 | 607 | 900 | 675 | 808 |
| | 10 | 643 | 619 | 904 | 951 | 719 | 813 |
| | 10 | 604 | 715 | 811 | 786 | 2,119 | 1,123 |
| 101 | 0 | 781 | 697 | 671 | 884 | 753 | 1,003 |
| | 10 | 685 | 1,028 | 683 | 871 | 726 | 847 |
| | 10 | 684 | 626 | 694 | 785 | 731 | 1,018 |
| 102 | 0 | 665 | 698 | 725 | 1,303 | 765 | 787 |
| | 10 | 998 | 1,044 | 758 | 791 | 716 | 923 |
| | 10 | 643 | 874 | 674 | 732 | 698 | 967 |
| 103 | 0 | 937 | 717 | 693 | 1,449 | 859 | 784 |
| | 10 | 7045 | 816 | 762 | 994 | 760 | 1,120 |
| | 10 | 6,580 | 1,159 | 3,574 | 807 | 1,090 | 1,383 |
| 104 | 0 | 681 | 749 | 714 | 991 | 5,096 | 778 |
| | 10 | 1,002 | 624 | 754 | 851 | 1,272 | 824 |
| | 10 | 795 | 668 | 717 | 795 | 998 | 1,272 |

48 different primer combinations were tested in total. Seven of the combinations tested gave at least one positive result at 10 copies per reaction.

### Example 3

### Testing POL Specific Detection Probes on In Vitro Transcribed RNA Designed to Detect Amplicons from MLV

The purpose of this experiment was to test the POL specific detection probes (SEQ ID Nos. 148, 149, and 150) for their ability to detect amplified in vitro transcribed RNA (SEQ ID NO:96). Primers consisting of SEQ ID Nos. 97 and 110 were used to amplify the in vitro transcribed RNA. The procedures and oligonucleotide concentrations were the same as those in Example 2, however, 0, 10, and 100 copies of in vitro transcribed RNA were used per reaction.

The results are shown below in Table 2, representing the RLU obtained for each reaction.

TABLE 2
Detection Probes

| SEQ ID NO:/BP* | Copies/Rxn | Ave. RLU | % Pos. |
|---|---|---|---|
| 148/9, 10 | 0 | 2,219 | 0 |
| | 10 | 648,471 | 40 |
| | 100 | 3,471,200 | 100 |
| 148/12, 13 | 0 | 5,852 | 20 |
| | 10 | 861,900 | 60 |
| | 100 | 2,117,377 | 80 |
| 149/9, 10 | 0 | 5,032 | 0 |
| | 10 | 151,229 | 80 |
| | 100 | 1,471,021 | 100 |
| 149/17, 18 | 0 | 61,522 | 0 |
| | 10 | 9,202 | 20 |
| | 100 | 2,004,193 | 100 |
| 150/7, 8 | 0 | 894 | 0 |
| | 10 | 250,406 | 80 |
| | 100 | 757,589 | 100 |
| 150/12, 13 | 0 | 2,510 | 0 |
| | 10 | 697,193 | 80 |
| | 100 | 7,807,680 | 100 |

*BP is the base position between which the AE is incorporated

All probes were able to detect at 100% the amplicons at 100 copies per reaction. Three probes were able to detect at 80% at 10 copies per reaction.

### Example 4

### Further Evaluation of the POL Specific Primers Designed to Amplify MLV

Four amplification systems were selected to investigate the sensitivity of the system. The first system comprised SEQ ID Nos. 97 and 110, the second system comprised SEQ ID Nos. 99 and 110, the third system comprised SEQ ID Nos. 97, 110, and 114, and the fourth system comprised SEQ ID Nos. 97, 103, 110, and 114. All amplification systems used SEQ ID NO:150 (AE incorporated between bases 7 and 8) as the detection probe. This assay was set-up and performed generally as described in Example 2, however, 0, 5, 10, 100, and 1000 copies of in vitro transcribed RNA (SEQ ID NO:96) were used per reaction.

The results are shown below in Table 3, representing the RLU obtained for each reaction.

TABLE 3
Amplification system

| SEQ ID Nos. | Copies/Rxn | Ave. RFU | % Pos. |
|---|---|---|---|
| 97 and 110 | 0 | 600 | 0 |
| | 5 | 180,367 | 40 |
| | 10 | 204,736 | 100 |
| | 100 | 331,958 | 100 |
| | 1,000 | 449,353 | 100 |
| 99 and 110 | 0 | 474 | 0 |
| | 5 | 299,820 | 40 |
| | 10 | 732,000 | 100 |
| | 100 | 614,177 | 100 |
| | 1,000 | 746,406 | 100 |
| 97, 110, and | 0 | 613 | 0 |
| 114 | 5 | 3,639 | 20 |
| | 10 | 180,118 | 100 |
| | 100 | 198,561 | 100 |
| | 1,000 | 546,078 | 100 |
| 97, 103, 110, | 0 | 1,832 | 0 |
| and 114 | 5 | 58,511 | 60 |
| | 10 | 309,674 | 100 |
| | 100 | 199,446 | 100 |
| | 1,000 | 579,062 | 100 |

All amplification systems were able to detect 10 copies per reaction at 100%. Having four amplification primers increased the sensitivity from detecting 5 copies per reaction at 40% to 60%.

### Example 5

### Testing POL Capture Probes Designed to Capture MLV Nucleic Acid

Three different target capture probes (SEQ ID Nos. 134, 135, and 136) were investigated for use in the MLV POL assay. The capture probes were tested individually and in combination with each other. Target capture was performed substantially as follows (described in detail in U.S. Pat. No. 6,110,678). In vitro transcribed RNA (SEQ ID NO:96) was spiked at 0, 5, and 50 copies per reaction into target capture reagent containing HEPES, LiOH, lithium lauryl sulfate (LLS), succinate, 5 μmol/reaction one of capture probes SEQ ID NOS:134, 135, 136 or a combination of the fore mentioned sequences, and magnetic particles attached to a poly-dT14 immobilization probe. Target capture hybridization occurred in this reaction mixture by incubating the mixture at a first temperature (60.deg. C.), allowing the capture oligomer to bind specifically to its complementary target sequence. Then, the mixture was cooled to 40.deg. C. or lower (e.g., room temperature) to allow the 3′ tail of the capture oligomer to hybridize to its complementary oligomer on the particle. Following the second hybridization, the mixture was treated to separate the solid support with its bound complex of nucleic acids from the other components in the mixture, e.g., by using magnetic separation. Generally, separation employed a rack containing a magnet to pull the magnetic particles with bound nucleic acid complexes to the side of the tube. Then the supernatant was removed and the bound complexes on the particles were washed with a washing buffer containing HEPES, NaOH, EDTA, absolute ethanol, methyl paraben, propyl paraben, NaCl, and sodium dodecyl sulfate (SDS) by suspending the magnetic particles in washing buffer, separating particles to the tube side, and removing the supernatant. After target capture, amplification and detection was performed on each sample using SEQ ID Nos. 97, 110, and 150 (AE incorporated between bases 7 and 8). The amplification and detection was set-up and performed generally as described in Example 2, minus the addition of additional in vitro transcribed RNA. Each reaction was performed 10 times.

The results are shown in the following Table 4:

TABLE 4
Capture Probe Analysis (RLU)

| SEQ ID NO: | Copies/Rxn | Ave. RLU | % Pos. |
|---|---|---|---|
| 134 | 0 | 374 | 0 |
| | 5 | 25,751 | 50 |
| | 50 | 122,040 | 90 |
| 135 | 0 | 388 | 0 |
| | 5 | 111,070 | 50 |
| | 50 | 166,961 | 90 |
| 136 | 0 | 432 | 0 |
| | 5 | 142,901 | 50 |
| | 50 | 204,895 | 90 |
| 134 and 135 | 0 | 630 | 0 |
| | 5 | 25,850 | 60 |
| | 50 | 79,401 | 100 |
| 134 and 136 | 0 | 510 | 0 |
| | 5 | 14,196 | 80 |
| | 50 | 41,598 | 100 |
| 135 and 136 | 0 | 403 | 0 |
| | 5 | 32,458 | 70 |
| | 50 | 105,197 | 100 |
| 135, 135, | 0 | 986 | 0 |
| and 136 | 5 | 145,862 | 50 |
| | 50 | 172,840 | 100 |

The performance of each target capture probe was similar for the percent positive, however, the average RLU was higher at the 5 copies/reaction level for SEQ ID Nos. 135 and 136 than for SEQ ID NO: 134. Using multiple target capture probes works better than using one.

### Example 6

### Testing POL Assay Using Spiked Clinical Samples and Using Oligomers Designed to Amplify and Detect MLV

The purpose of this experiment was to test the POL specific target capture probes (SEQ ID Nos. 135 and 136), amplification primers (SEQ ID Nos. 97 and 110), and detection probe (SEQ ID NO:148 AE incorporated between bases 12 and 13) for their ability to capture, amplify, and detect in vitro transcribed RNA (SEQ ID NO:96) spiked into blood samples. Whole blood, centrifuged whole blood and buffy coat samples were collected from donors. Two aliquots were taken from each donor sample and one aliquot was spiked with 200 copies of in vitro transcript. The samples underwent target capture using the procedures and oligonucleotide concentrations described in Example 5, followed by amplification and detection using the procedures and oligonucleotide concentrations described in Example 2.

The results are shown in the following Table 5:

TABLE 5
Spiked Clinical Samples (RLU)

| Sample | RLU | Spiked RLU |
|---|---|---|
| Buffy Coat 1 | 908 | 48,281 |
| Buffy Coat 2 | 1,340 | 154,711 |
| Buffy Coat 3 | 1,348 | 138,860 |
| Buffy Coat 4 | 1,932 | 187,527 |
| Buffy Coat 5 | 3,085 | 302,025 |
| Buffy Coat 6 | 1,846 | 190,694 |
| Buffy Coat 7 | 1,235 | 71,801 |
| Buffy Coat 8 | 889 | 87,702 |
| Buffy Coat 9 | 1,126 | 167,748 |
| Buffy Coat 10 | 1,215 | 670,876 |
| Buffy Coat 11 | 1,401 | 82,145 |
| Buffy Coat 12 | 1,590 | 1,137,543 |
| Whole Blood 1 | 5,272 | 223,929 |
| Whole Blood 2 | 1,066 | 918,009 |
| Whole Blood 3 | 1,971 | 516,139 |
| Whole Blood 4 | 1,334 | 585,220 |
| Whole Blood 5 | 1,293 | 618,985 |
| Whole Blood 6 | 1,443 | 1,202 |
| Whole Blood 7 | 891 | 260,466 |
| Whole Blood 8 | 779 | 659,003 |
| Whole Blood 9 | 808 | 846,344 |
| Whole Blood 10 | 1,193 | 996,986 |
| Whole Blood 11 | 863 | 400,121 |
| Whole Blood 12 | 974 | 513,186 |
| Whole Blood 13 | 1,227 | 460,765 |
| Whole Blood 14 | 1,048 | 676,125 |
| Whole Blood 15 | 1,350 | 193,062 |
| Whole Blood 16 | 148,380 | 360,007 |
| Whole Blood 17 | 806 | 283,479 |
| Whole Blood 18 | 797 | 186,807 |
| Whole Blood 19 | 1,068 | 305,836 |
| Whole Blood 20 | 870 | 588,208 |
| Whole Blood 21 | 950 | 437,776 |
| Whole Blood 22 | 1,738 | 895,805 |
| Whole Blood CFS 1 | 1,833 | 1,094,436 |
| Whole Blood CFS 2 | 923 | 848,832 |
| Whole Blood CFS 3 | 905 | 581,301 |
| Whole Blood CFS 4 | 859 | 847,832 |
| Whole Blood CFS 5 | 893 | 730,968 |
| Whole Blood CFS 6 | 840 | 430,121 |
| Whole Blood CFS 7 | 1,193 | 1,292,031 |
| Whole Blood CFS 8 | 1,790 | 314,454 |
| Whole Blood CFS 9 | 862 | 382,602 |
| Whole Blood CFS 10 | 1,539 | 200,786 |

The capture probes, amplification primers, and detection probe were able to detect the in vitro transcript spiked into blood.

### Example 7

### Testing LTR Specific Primers in In Vitro Transcribed RNA Designed to Detect MLV

The purpose of this experiment was to test the LTR specific primers for their ability to detect and amplify in vitro transcribed RNA (SEQ ID NO:95). Primers consisting of SEQ ID Nos. 105 to 109 (Non-T7) and SEQ ID Nos. 116 to 121 (T7) were tested.

The amplification mixture used contained 5 pmol of T7 primer and 5 pmol of nonT7 primer per reaction. 0 or 20 copies of in vitro transcribed RNA (SEQ ID NO:95) was used per reaction. 200 microlitres of oil was added to each tube and vortexed, followed by incubation at 62.deg. C. for 10 minutes followed by 41.5.deg. C. for 20 minutes. 25 microlitres of enzyme was added to each tube, hand shaken and the incubation continued at 41.5.deg. C. for 50 minutes. 100 microlitres of probe reagent (AE labeled SEQ ID No. 151 at 2.5E6 RLU/100 ul of hybridizing reagent) was added, vortexted and incubated at 62.deg. C. for 20 minutes. 250 microlitres of selection reagent was then added and the tube vortexed, followed by incubation at 62.deg. C. for 10 minutes. Detect reagent I and II were added immediately before measuring the relative light units (RLU).

The results are shown below in Table 6, representing the RLU obtained for each reaction Positive results are indicated in bold.

TABLE 6
Amplification Oligomer Combinations

| SEQ | | | | | | | |
|---|---|---|---|---|---|---|---|
| ID | | | | | | | Copies/ |
| No. | 116 | 117 | 118 | 119 | 120 | 121 | RXN |
| 105 | 272 | 339 | 245 | 589 | 982 | 368 | 0 |
| | 13,525 | 15,273 | 8,343 | 1,656 | 8,793 | 299 | 20 |
| | 39,159 | 11,961 | 7,956 | 612 | 1,017 | 432 | 20 |
| 106 | 310 | 298 | 238 | 699 | 1,017 | 1,507 | 0 |
| | 1,252 | 2,032 | 9,621 | 1,844 | 3,963 | 350 | 20 |
| | 9,952 | 12,488 | 8,495 | 468 | 1,933 | 481 | 20 |
| 107 | 281 | 254 | 362 | 333 | 400 | 1,485 | 0 |
| | 497,147 | 179,462 | 160,735 | 420 | 1,533 | 1,341 | 20 |
| | 187,466 | 271,406 | 94,368 | 508 | 6,133 | 1,150 | 20 |
| 108 | 341 | 334 | 772 | 467 | 1,508 | 1,019 | 0 |
| | 348,769 | 135,721 | 674,018 | 355,744 | 195,367 | 228,769 | 20 |
| | 414,104 | 166,126 | 692,580 | 4,375 | 206,003 | 167,338 | 20 |
| 109 | 367 | 484 | 348 | 1,255 | 330 | 418 | 0 |
| | 341,818 | 140,139 | 653,470 | 1,213 | 327,406 | 81,366 | 20 |
| | 382,251 | 259,637 | 550,166 | 988 | 353,198 | 38,617 | 20 |

30 different primer combinations were tested in total. Fifteen of the combinations tested gave positive results at 20 copies per reaction.

### Example 8

### Testing LTR Capture Probes Designed to Capture MLV

Four different target capture probes (SEQ ID Nos. 137, 138, 139, and 140) were investigated for use in the MLV LTR assay. Target capture was performed substantially as follows (described in detail in U.S. Pat. No. 6,110,678). In vitro transcribed RNA (SEQ ID NO:95) was spiked at 0, 10, and 200 copies per reaction into target capture reagent containing HEPES, LiOH, lithium lauryl sulfate (LLS), succinate, 5 ppmol/reaction one of capture probes SEQ ID Nos. 137, 138, 139, 140, or a combination of SEQ ID NO:140 with SEQ ID Nos. 137, 138, or 139, and magnetic particles attached to a poly-dT14 immobilization probe. Target capture hybridization occurred in this reaction mixture by incubating the mixture at a first temperature (60.deg. C.), allowing the capture oligomer to bind specifically to its complementary target sequence. Then, the mixture was cooled to 40.deg. C. or lower (e.g., room temperature) to allow the 3′ tail of the capture oligomer to hybridize to its complementary oligomer on the particle. Following the second hybridization, the mixture was treated to separate the solid support with its bound complex of nucleic acids from the other components in the mixture, e.g., by using magnetic separation. Generally, separation employed a rack containing a magnet to pull the magnetic particles with bound nucleic acid complexes to the side of the tube. Then the supernatant was removed and the bound complexes on the particles were washed with a washing buffer containing HEPES, NaOH, EDTA, absolute ethanol, methyl paraben, propyl paraben, NaCl, and sodium dodecyl sulfate (SDS) by suspending the magnetic particles in washing buffer, separating particles to the tube side, and removing the supernatant. After target capture, amplification and detection was performed using two different sets of amplification primers. The first set consisted of SEQ ID Nos. 108 and 116 and the second set consisted of SEQ ID Nos. 108 and 118. Both sets of amplification primers were used with the same detection probe (SEQ ID NO:154 with an AE incorporated between bases 6 and 7). The amplification and detection was set-up and performed generally as described in Example 2, minus the addition of additional in vitro transcribed RNA. Each reaction was performed 5 times for the 0 and 200 copies/reaction and 10 times for the 10 copies/reaction.

The results are shown in the following Table 7:

TABLE 7
Individual Capture Probe Analysis (RLU)

| Primers | Target Capture | Copies/Rxn | Ave. RLU | % Pos. |
|---|---|---|---|---|
| SEQ ID Nos. | SEQ ID NO: | 0 | 733 | 0 |
| 108 and 116 | 137 | 10 | 1,679 | 30 |
| | | 200 | 167,379 | 100 |
| | SEQ ID NO: | 0 | 593 | 0 |
| | 138 | 10 | 150,841 | 40 |
| | | 200 | 277,157 | 80 |
| | SEQ ID NO: | 0 | 631 | 0 |
| | 139 | 10 | 29,938 | 30 |
| | | 200 | 350,772 | 100 |
| | SEQ ID NO: | 0 | 788 | 0 |
| | 140 | 10 | 109,354 | 80 |
| | | 200 | 637,366 | 100 |
| SEQ ID Nos. | SEQ ID NO: | 0 | 761 | 0 |
| 108 and 118 | 137 | 10 | 201,603 | 20 |
| | | 200 | 565,619 | 80 |
| | SEQ ID NO: | 0 | 824 | 0 |
| | 138 | 10 | 231,052 | 20 |
| | | 200 | 563,457 | 100 |
| | SEQ ID NO: | 0 | 1,125 | 0 |
| | 139 | 10 | 55,904 | 10 |
| | | 200 | 78,135 | 40 |
| | SEQ ID NO: | 0 | 761 | 0 |
| | 140 | 10 | 504,317 | 80 |
| | | 200 | 655,100 | 100 |

SEQ ID NO:140 had a higher percent positive at 10 copies/reaction than the other target capture probes. The above experiment was repeated this time comparing SEQ ID NO:140 alone to SEQ ID NO:140 combined with an additional target capture probe (i.e. SEQ ID Nos. 137, 138, or 139). The amplification primer sets were (1) SEQ ID Nos.

The results are shown in the following Table 8:

TABLE 8
Combination Capture Probe Analysis

| Primers | Target Capture | Copies/Rxn | Ave. RLU | % Pos. |
|---|---|---|---|---|
| SEQ ID Nos. | SEQ ID NO: | 0 | 453 | 0 |
| 108 and 118 | 140 | 10 | 146360 | 30 |
| | | 200 | 804153 | 100 |
| | SEQ ID Nos. | 0 | 471 | 0 |
| | 140 and 137 | 10 | 141997 | 50 |
| | | 200 | 680575 | 100 |
| | SEQ ID Nos. | 0 | 890 | 0 |
| | 140 and 138 | 10 | 371650 | 80 |
| | | 200 | 853812 | 100 |
| | SEQ ID Nos. | 0 | 470 | 0 |
| | 140 and 139 | 10 | 111229 | 40 |
| | | 200 | 896227 | 100 |
| SEQ ID Nos. | SEQ ID NO: | 0 | 641 | 0 |
| 108, 118, and | 140 | 10 | 40079 | 70 |
| 120 | | 200 | 100927 | 100 |
| | SEQ ID Nos. | 0 | 497 | 0 |
| | 140 and 137 | 10 | 46040 | 50 |
| | | 200 | 124124 | 100 |
| | SEQ ID Nos. | 0 | 529 | 0 |
| | 140 and 138 | 10 | 15613 | 50 |
| | | 200 | 223157 | 100 |
| | SEQ ID Nos. | 0 | 550 | 0 |
| | 140 and 139 | 10 | 16167 | 60 |
| | | 200 | 122983 | 100 |

### Example 9

### Exemplary Sequences

| SEQ ID NO: | Sequence 5′ to 3′ |
|---|---|
| 49 | AATTTAATACGACTCACTATAGGGAGA |
| 72 | TTTAAAAAAAAAAAAAAAAAAAAAAAAAAAAAA |
| 85 | See GenBank Accession Number EF185282.1 GI: 121104176 |
| | entered at NCBI on Jan. 10, 2007 |
| 97 | ACCCTGCCAGTCCCCCTGGAA |
| 98 | ACCCTGCCAGTCCCCCTGGA |
| 99 | ACCCTGCCAGTCCCCCTG |
| 100 | GGGAATACTGGTACCCTGCCAGTC |
| 101 | GGGAATACTGGTACCTTGCCAGTC |
| 102 | GAATACTGGTACCTTGCCAGTC |
| 103 | AAGCCCCACATACAGAG |
| 104 | CAAGCCCCACATACAGAG |
| 105 | CGTGAATAAAAGATTTTATTCAG |
| 106 | CGTGAATAAAAGATTTTATTCAGT |
| 107 | CACGTGAATAAAAGATTTTATTC |
| 108 | GATTTTATTCAGTTTCCAGAAAGAGG |
| 109 | GATTTTATTCAGTTTCCAGAAAG |
| 110 | AATTTAATACGACTCACTATAGGGAGATCCACCCGCTTGTTGACTTCT |
| 111 | AATTTAATACGACTCACTATAGGGAGACACCCGCTTGTTGACTTCTCT |
| 112 | AATTTAATACGACTCACTATAGGGAGATGTCTTCCACCCGCTTGTT |
| 113 | AATTTAATACGACTCACTATAGGGAGATGTCTTCCACCCGCTTGT |
| 114 | AATTTAATACGACTCACTATAGGGAGAGTTGGGCACGGTGGGGTGG |
| 115 | AATTTAATACGACTCACTATAGGGAGAGTTGGGCACGGTGGGGTG |
| 116 | AATTTAATACGACTCACTATAGGGAGAATGCCTTGCAAAATGGCGTTACTG |
| 117 | AATTTAATACGACTCACTATAGGGAGAATGCCTTGCAAAATGGCGTTACT |
| 118 | AATTTAATACGACTCACTATAGGGAGAATGCCTTGCAAAATGGCGTTAC |
| 119 | AATTTAATACGACTCACTATAGGGAGATTTTCCATGCCTTGCAA |
| 120 | AATTTAATACGACTCACTATAGGGAGAGAACTCAGCTCTGGTA |
| 121 | AATTTAATACGACTCACTATAGGGAGATGAGAACTCAGCTCTGGT |
| 122 | TCCACCCGCTTGTTGACTTCT |
| 123 | CACCCGCTTGTTGACTTCTCT |
| 124 | TGTCTTCCACCCGCTTGTT |
| 125 | TGTCTTCCACCCGCTTGT |
| 126 | GTTGGGCACGGTGGGGTGG |
| 127 | GTTGGGCACGGTGGGGTG |
| 128 | ATGCCTTGCAAAATGGCGTTACTG |
| 129 | ATGCCTTGCAAAATGGCGTTACT |
| 130 | ATGCCTTGCAAAATGGCGTTAC |
| 131 | TTTTCCATGCCTTGCAA |
| 132 | GAACTCAGCTCTGGTA |
| 133 | TGAGAACTCAGCTCTGGT |
| 134 | UGUGACAUGGGGUAUUGUUUUAUGGATTTAAAAAAAAAAAAAAAAAAAAAA |
| | AAAAAAAA |
| 135 | UGGCUUCUUGUGACAUGGGGUAUUGUUTTAAAAAAAAAAAAAAAAAAAAAA |
| | AAAAAAAA |
| 136 | CUCAAAGGCGAAGAGAGGCUGACUGGTTTAAAAAAAAAAAAAAAAAAAAAA |
| | AAAAAAAA |
| 137 | AUCUGUUCUUGGCCCUGAGCTTTAAAAAAAAAAAAAAAAAAAAAAAAAAAA |
| | AA |
| 138 | ACCAUCUGUUCUUGGCCCUGAGCTTTAAAAAAAAAAAAAAAAAAAAAAAAA |
| | AAAAA |
| 139 | ACUAUCUGUUCUUGGCCCUGAGCTTTAAAAAAAAAAAAAAAAAAAAAAAAA |
| | AAAAA |
| 140 | UACAGAAGCGAGAAGCGAGCUGTTTAAAAAAAAAAAAAAAAAAAAAAAAAA |
| | AAAA |
| 141 | UGUGACAUGGGGUAUUGUUUUAUGGA |
| 142 | UGGCUUCUUGUGACAUGGGGUAUUGUU |
| 143 | CUCAAAGGCGAAGAGAGGCUGACUGG |
| 144 | AUCUGUUCUUGGCCCUGAGC |
| 145 | ACCAUCUGUUCUUGGCCCUGAGC |
| 146 | ACUAUCUGUUCUUGGCCCUGAGC |
| 147 | UACAGAAGCGAGAAGCGAGCUG |
| 148 | CUACCCGUUAAGAAACCAGG |
| 149 | CUACCCGUUAAGAAACCAGGGACUAA |
| 150 | CGUUAAGAAACCAGGGACUAA |
| 151 | CAGAAAGAGGGGGGAATG |
| 152 | CAGAAAGAGGGIGGAAUG |
| 153 | CAUUCCCCCCUCUUUCUG |
| 154 | GGAAUGAAAGACCCC |
| 155 | GACCCCACCAUAAGGCUUAG |
| 156 | GACCCCUUCAUAAGGCUUAG |
| 157 | CACCCGCTTGTTGACTTCT |
| 158 | CGTGAATAAAAGATTTTATTC |
| 159 | GAACTCAGCTCTGGT |

SEQ ID NOS:1-24 and 110-121 are T7 promoter based amplification oligomers; SEQ ID NOS:25-48, 50-65, 97-109, and 122-133 are amplification oligomer target binding sequences; SEQ ID NO:49 is a T7 promoter sequence; SEQ ID NOS:66-67 and 148-156 are probe target binding sequences; SEQ ID NOS:68-71, 77-80, and 134-140 are target capture oligomers; SEQ ID NO:72 is a capture region of a target capture oligomer; SEQ ID NOS:73-76, 81-84, and 141-147 are target binding sequences for target capture oligomers; SEQ ID NOS: 86-93, 99, 102, 103, 109, 125, 127, 130, 131, and 157-159 are consensus sequences for amplification oligomer target binding sequences; SEQ ID NO:94 is a consensus sequence for target capture oligomer target binding sequences.

IVT sequence (LTR)
SEQ ID NO: 95
GGGCGAAUUGGGUACCGAUAUUGGAGAUGGUUGCCGCUCUCCCGGGGGAAGAAAAAGGACAAGACUAUAUGAUUU
CUAUGUUUGCCCCGGUCAUACUGUAUUAACAGGGUGUGGAGGGCCGAGAGAGGGCUACUGUGGCAAAUGGGGAUG
UGAGACCACUGGACAGGCAUACUGGAAGCCAUCAUCAUCAUGGGACCUAAUUUCCCUUAAGCGAGGAAACACUCC
UAAGGGUCAGGGCCCCUGUUUUGAUUCCUCAGUGGGCUCCGGUAGCAUCCAGGGUGCCACACCGGGGGGUCGAUG
CAACCCCCUAGUCCUAGAAUUCACUGACGCGGGUAAAAGGGCCAGCUGGGAUGCCCCCAAAACAUGGGGACUAAG
ACUGUAUCGAUCCACUGGGGCCGACCCGGUGACCCUGUUCUCUCUGACCCGCCAGGUCCUCAAUGUAGGGCCCCG
CGUCCCCAUUGGGCCUAAUCCCGUGAUCACUGAACAGCUACCCCCCUCCCAACCCGUGCAGAUCAUGCUCCCCAG
GCCUCCUCGUCCUCCUCCUUCAGGCGCGGCCUCUAUGGUGCCUGGGGCUCCCCCGCCUUCUCAACAACCUGGGAC
GGGAGACAGGCUGCUAAACCUGGUAGAAGGAGCCUACCAAGCCCUCAACCUCACCAGUCCCGACAAAACCCAAGA
GUGCUGGCUGUGUCUAGUAUCGGGACCCCCCUACUACGAAGGGGUGGCCGUCCUAGGUACUUACUCCAACCAUAC
CUCUGCCCCGGCUAACUGCUCCGUGACCUCCCAACACAAGCUGACCCUGUCCGAAGUGACCGGGCAGGGACUCUG
CAUAGGAGCAGUUCCCAAAACCCAUCAGGCCCUGUGUAAUACCACCCAGAAGACGAGCGACGGGUCCUACUAUUU
GGCCUCUCCCGCCGGGACCAUUUGGGCUUGCAGCACCGGGCUCACUCCCUGUCUAUCUACUACUGUGCUUAACUU
AACCACUGAUUACUGUGUCCUGGUUGAACUCUGGCCAAAGGUAACCUACCACUCCCCUAAUUAUGUUUAUGGCCA
GUUUGAAAAGAAAACUAAAUAUAAAAGAGAGCCGGUGUCAUUAACUCUGGCCCUGCUGUUGGGAGGACUUACUAU
GGGCGGCAUAGCUGCAGGAGUUGGAACAGGGACUACAGCCCUAGUGGCCACCAAACAAUUCGAGCAGCUCCAGGC
AGCCAUACAUACAGACCUUGGGGCCUUAGAAAAAUCAGUCAGUGCCCUAGAAAAGUCUCUGACCUCGUUGUCUGA
GGUGGUCCUACAGAACCGGAGGGGAUUAGAUCUACUGUUCCUAAAAGAAGGAGGAUUAUGUGCUGCCCUAAAAGA
AGAAUGCUGUUUUUACGCGGACCACACUGGCGUAGUAAGAGAUAGCAUGGCAAAGCUAAGAGAAAGGUUAAACCA
GAGACAAAAAUUGUUCGAAUCAGGACAAGGGUGGUUUGAGGGACUGUUUAACAGGUCCCCAUGGUUCACGACCCU
GAUAUCCACCAUUAUGGGCCCUCUGAUAGUACUUUUAUUAAUCCUACUCUUCGGACCCUGUAUUCUCAACCGCUU
GGUCCAGUUUGUAAAAGACAGAAUUUCGGUAGUGCAGGCCCUGGUUCUGACCCAACAGUAUCACCAACUCAAAUC
AAUAGAUCCAGAAGAAGUGGAAUCACGUGAAUAAAAGAUUUUAUUCAGUUUCCAGAAAGAGGGGGGAAUGAAAGA
CCCCACCAUAAGGCUUAGCACGCUAGCUACAGUAACGCCAUUUUGCAAGGCAUGGAAAAGUACCAGAGCUGAGUU
CUCAAAAGUUACAAGGAAGUUUAAUUAAAGAAUAAGGCUGAAUAACACUGGGACAGGGGCCAAACAGGAUAUCUG
UAGUCAGGCACCUGGGCCCCGGCUCAGGGCCAAGAACAGAUGGUCCUCAGAUAAAGCGAAACUAACAACAGUUUC
UGGAAAGUCCCACCUCAGUUUCAAGUUCCCCAAAAGACCGGGAAAUACCCCAAGCCUUAUUUAAACUAACCAAUC
AGCUCGCUUCUCGCUUCUGUACCCGCGCUUUUUGCUCCCCAGUCCUAGCCCUAUAAAAAAGGGGUAAGAACUCCA
CACUCGGCGCGCCAGUCAUCCGAUAGACUGAGAAGCUUGAUAUCGAAUUCCUGCAGCCCGGGGGAUCCACUAGUU
CUAGAGCGGCC
IVT sequence (POL)
SEQ ID NO: 96
GGGCGAAUUGGGUACCGAUACAGACCGGGUUCAGUUCGGACCGGUGGUGGCCCUCAACCCGGCCACCCUGCUCCC
CCUACCGGAAAAGGAAGCCCCCCAUGACUGCCUCGAGAUCUUGGCUGAGACGCACGGAACCAGACCGGACCUCAC
GGACCAGCCCAUCCCAGACGCUGAUUACACUUGGUACACAGAUGGAAGCAGCUUCCUACAAGAAGGACAACGGAG
AGCUGGAGCAGCGGUGACUACUGAGACCGAGGUAAUCUGGGCGAGGGCUCUGCCGGCUGGAACAUCCGCCCAACG
AGCCGAACUGAUAGCACUCACCCAAGCCUUAAAGAUGGCAGAAGGUAAGAAGCUAAAUGUUUACACUGAUAGCCG
CUAUGCCUUCGCCACGGCCCAUGUCCAUGGAGAAAUAUAUAGGAGGCGAGGGUUGCUGACCUCAGAAGGCAGAGA
AAUUAAAAACAAGAACGAGAUCUUGGCCUUGCUAAAAGCUCUCUUUCUGCCCAAACGACUUAGUAUAAUUCACUG
UCCAGGACAUCAAAAAGGAAACAGUGCUGAGGCCAGAGGCAACCGUAUGGCAGAUCAAGCAGCCCGAGAGGCAGC
CAUGAAGGCAGUUCUAGAAACCUCUACACUCCUCAUAGAGGACUCAACCCCGUAUACGCCUCCCCAUUUCCAUUA
CACCGAAACAGAUCUCAAAAGACUACGGGAACUGGGAGCCACCUACAAUCAGACAAAAGGAUAUUGGGUCCUACA
AGGCAAACCUGUGAUGCCCGAUCAGUCCGUGUUUGAACUGUUAGACUCCCUACACAGACUCACCCAUCUGAGCCC
UCAAAAGAUGAAGGCACUCCUCGACAGAGAAGAAAGCCCCUACUACAUGUUAAACCGGGACAGAACUAUCCAGUA
UGUGACUGAGACCUGCACCGCCUGUGCCCAAGUAAAUGCCAGCAAAGCCAAAAUUGGGGCAGGGGUGCGAGUACG
CGGACAUCGGCCAGGCACCCAUUGGGAAGUUGAUUUCACGGAAGUAAAGCCAGGACUGUAUGGGUACAAGUACCU
CCUAGUGUUUGUAGACACCUUCUCUGGCUGGGUAGAGGCAUUCCCGACCAAGCGGGAAACUGCCAAGGUCGUGUC
CAAAAAGCUGUUAGAAGACAUUUUUCCGAGAUUUGGAAUGCCGCAGGUAUUGGGAUCUGAUAACGGGCCUGCCUU
CGCCUCCCAGGUAAGUCAGUCAGUGGCCGAUUUACUGGGGAUCGAUUGGAAGUUACAUUGUGCUUAUAGACCCCA
GAGUUCAGGACAGGUAGAAAGAAUGAAUAGAACAAUUAAGGAGACUUUGACCAAAUUAACGCUUGCAUCUGGCAC
UAGAGACUGGGUACUCCUACUCCCCUUAGCCCUCUACCGAGCCCGGAAUACUCCGGGCCCCCACGGACUGACUCC
GUAUGAAAUUCUGUAUGGGGCACCCCCGCCCCUUGUCAAUUUUCAUGAUCCUGAAAUGUCAAAGUUAACUAAUAG
UCCCUCUCUCCAAGCUCACUUACAGGCCCUCCAAGCAGUACAACAAGAGGUCUGGAAGCCGCUGGCCGCUGCUUA
UCAGGACCAGCUAGAUCAGCCAGUGAUACCACACCCCUUCCGUGUCGGUGACGCCGUGUGGGUACGCCGGCACCA
GACUAAGAACUUAGAACCUCGCUGGAAAGGACCCUACACCGUCCUGCUGACAACCCCCACCGCUCUCAAAGUAGA
CGGCAUCUCUGCGUGGAUACACGCCGCUCACGUAAAGGCGGCGACAACUCCUCCGGCCGGAACAGCAUGGAAAGU
CCAGCGUUCUCAAAACCCCUUAAAGAUAAGAUUAACCCGUGGGGCCCCCUGAUAAUUAUGGGGAUCUUGGUGAGG
GCAGGAGCCUCAGUACAACGUGACAGCCCUCACCAGGUCUUUAAUGUCACUUGGAAAAUUACCAACCUAAUGACA
GGACAAACAGCUAAUGCUACCUCCCUCCUGGGGACGAUGACAGACACUUUCCCUAAACUAUAUUUUGACUUGUGU
GAUUUAGUUGGAGACAACUGGGAUGACCCGGAACCCGAUAUUGGAGAUGGUUGCCGCUCUCCCGGGGGAAGAAAA
AGGACAAGACUAUAUGAUUUCUAUGUUUGCCCCGGUCAUACUGUAUUAACAGGGUGUGGAGGGCCGAGAGAGGGC
UACUGUGGCAAAUGGGGAUGUGAGACCACUGGACAGGCAUACUGGAAGCCAUCAUCGGAAUU

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the invention claimed. Thus, it should be understood that although the present invention has been specifically disclosed by preferred embodiments and optional features, modification and variation of the invention embodied therein herein disclosed may be resorted to by those skilled in the art, and that such modifications and variations are considered to be within the scope of this invention.

Other embodiments are within the following claims. In addition, where features or aspects of the methods are described in terms of Markush groups, those skilled in the art will recognize that the invention is also thereby described in terms of any individual member or subgroup of members of the Markush group.

1. A method for the amplification and identification of an MLV from a sample comprising the steps of:
(a) contacting a sample suspected of containing MLV with at least two amplification oligomers for generating an amplicon, wherein each of said at least two amplification oligomers is from about 10 to about 50 nucleobases in length and wherein said at least two amplification oligomers are respectively configured to specifically hybridize to regions within a target sequence of MLV selected from the group consisting of:
from residue 2800 to residue 2862 and from residue 2924 to residue 2971 of SEQ ID NO:85, or
from residue 7676 to residue 7713 and from residue 7756 to residue 7804 of SEQ ID NO:85;
(b) providing conditions sufficient for generating an amplicon from an MLV target nucleic acid present in said sample using said amplification oligomers from step a; and
(c) providing conditions for detecting said amplicon and determining whether said sample contains MLV target nucleic acid.

2. The method of claim 1, wherein at least one of said at least two amplification oligomers comprises a target binding sequence that contains a sequence selected from the group consisting of SEQ ID NOS: 99, 102, 103, 109, 125, 127, 130, 131, 157, 158, and 159.

3. The method of claim 1, wherein said at least two amplification oligomers comprise a first amplification oligomer comprising, consisting or consisting essentially of a target binding sequence containing SEQ ID NOS: 99, 102, and 103, and a second amplification oligomer comprising, consisting, or consisting essentially of a target binding sequence containing SEQ ID NOS: 125, 127, and 157.

4. The method of claim 1, wherein said at least two amplification oligomers comprise a first amplification oligomer comprising, consisting or consisting essentially of a target binding sequence containing SEQ ID NOS: 109 and 158, and a second amplification oligomer comprising, consisting, or consisting essentially of a target binding sequence containing SEQ ID NOS: 130, 131, and 159.

5. The method of claim 1, wherein said at least two amplification oligomers comprise a first amplification oligomer comprising, consisting or consisting essentially of a target binding sequence as set forth in any one of SEQ ID NOS: 97 to 104 and a second amplification oligomer comprising, consisting or consisting essentially of a target binding sequence as set forth in any one of SEQ ID NOS: 110 to 115 or 122 to 127.

6. The method of claim 1, wherein said at least two amplification oligomers comprise a first amplification oligomer comprising, consisting or consisting essentially of a target binding sequence as set forth in any one of SEQ ID NOS: 105 to 109 and a second amplification oligomer comprising, consisting or consisting essentially of a target binding sequence as set forth in any one of SEQ ID NOS: 116 to 121 or 128 to 133.

7. The method of claim 1, wherein said amplicon is detected using a detection probe oligomer comprising a target binding sequence set forth in one of SEQ ID NOS: 148 to 150.

8. The method of claim 1, wherein said amplicon is detected using a detection probe oligomer comprising a target binding sequence set forth in one of SEQ ID NO: 151 to 156.

9. A method for the multiplex amplification and identification of an MLV from a sample comprising the steps of:
(a) contacting a sample suspected of containing MLV with at least two amplification oligomer pairs for generating separate amplicons from an MLV target nucleic acid, wherein each amplification oligomer of said at least two amplification oligomer pairs is from 10 to about 50 nucleobases in length and
wherein a first amplification oligomer pair is configured to specifically hybridize to regions within a target sequence of MLV comprising, consisting or consisting essentially of from residue 2800 to residue 2862 and from residue 2924 to residue 2971 of SEQ ID NO:85, and
wherein a second amplification oligomer pair is configured to specifically hybridize to regions within a target sequence of MLV comprising, consisting or consisting essentially of from residue 7676 to residue 7713 and from residue 7756 to residue 7804 of SEQ ID NO:85;
(b) providing conditions sufficient for generating amplicons from an MLV target nucleic acid present in said sample using said amplification oligomers from step a;
(c) providing conditions for detecting said amplicon and determining whether said sample contained MLV target nucleic acid.

10. The method of claim 9, wherein
(i) said first amplification oligomer pair is one of SEQ ID NOS: 97 to 104 and one of SEQ ID NOS: 110 to 115 or 122 to 127 and
(ii) said second amplification oligomer pair is one of SEQ ID NOS: 105 to 109 and one of SEQ ID NOS: 116 to 121 or 128 to 133.

11. The method of claim 10, wherein said amplicon is detected using at least one detection probe oligomer comprising a target binding sequence as set forth in SEQ ID NO: 148 to 156.

12. The method of claim 9, wherein said amplicon is detected using a detection probe oligomer comprising a target binding sequence as set forth in SEQ ID NO: 148 to 156.

13. The method of claim 9, wherein said amplification reaction is substantially isothermal.

14. The method of claim 9, wherein said sample is or is derived from human blood.

15. A composition or a reaction mixture for use in an MLV target nucleic acid amplification assay comprising at least two amplification oligomers capable of stably hybridizing to MLV target nucleic acid,
wherein each amplification oligomer of said at least two amplification oligomers is from about 10 to about 50 nucleobases in length, and
wherein said at least two amplification oligomers are respectively configured to specifically hybridize to regions within a target sequence of MLV selected from the group consisting of:
from residue 2800 to residue 2862 and from residue 2924 to residue 2971 of SEQ ID NO:85; and
from residue 7676 to residue 7713 and from residue 7756 to residue 7804 of SEQ ID NO:85.

16. The composition or reaction mixture of claim 15, wherein at least one amplification oligomer of said at least two amplification oligomers comprises a target binding sequence that contains a sequence selected from the group consisting of SEQ ID NOS: 99, 102, 103, 109, 125, 127, 130, 131, 157, 158, and 159.

17. The composition or reaction mixture of claim 15, wherein said at least two amplification oligomers are one of SEQ ID NOS: 97 to 104; and one of SEQ ID NOS: 110 to 115 or 122 to 127.

18. The composition or reaction mixture of claim 15, wherein said at least two amplification oligomers are one of SEQ ID NOS: 105 to 109; and one of SEQ ID NOS: 116 to 121 or 128 to 133.

19. The composition or the reaction mixture of claim 15 for use in an MLV target nucleic acid multiplex amplification assay, wherein the at least two amplification oligomer pairs capable of stably hybridizing to an MLV target nucleic acid, comprise:
(i) a first amplification oligomer pair that is one of SEQ ID NOS: 97 to 104 and one of SEQ ID NOS: 110 to 115 or 122 to 127, and
(ii) a second amplification oligomer pair that is one of SEQ ID NOS: 105 to 109 and one of SEQ ID NOS: 116 to 121 or 128 to 133.

20. The composition or reaction mixture of claim 19, further including a detection probe oligomer wherein said detection probe oligomer comprises a target binding sequence set forth in one of SEQ ID NO: 148 to 156.


SEQUENCE LISTING (159 sequences)

SEQ ID NO: 1 - DNA, 50 bp, Artificial Sequence
  Synthetic Oligonucleotide
aatttaatacgactcactatagggagagtgccttcatcttttgagggctc

SEQ ID NO: 2 - DNA, 53 bp, Artificial Sequence
  Synthetic Oligonucleotide
aatttaatacgactcactatagggagaggagtgccttcatcttttgagggctc

SEQ ID NO: 3 - DNA, 49 bp, Artificial Sequence
  Synthetic Oligonucleotide
aatttaatacgactcactatagggagaggagtgccttcatcttttgagg

SEQ ID NO: 4 - DNA, 54 bp, Artificial Sequence
  Synthetic Oligonucleotide
aatttaatacgactcactatagggagagtcgaggagtgccttcatcttttgagg

SEQ ID NO: 5 - DNA, 53 bp, Artificial Sequence
  Synthetic Oligonucleotide
aatttaatacgactcactatagggagagtcgaggagtgccttcatcttttgag

SEQ ID NO: 6 - DNA, 55 bp, Artificial Sequence
  Synthetic Oligonucleotide
aatttaatacgactcactatagggagactgtcgaggagtgccttcatcttttgag

SEQ ID NO: 7 - DNA, 54 bp, Artificial Sequence
  Synthetic Oligonucleotide
aatttaatacgactcactatagggagagtagtaggggctttcttctctgtcgag

SEQ ID NO: 8 - DNA, 51 bp, Artificial Sequence
  Synthetic Oligonucleotide
aatttaatacgactcactatagggagagtaggggctttcttctctgtcgag

SEQ ID NO: 9 - DNA, 54 bp, Artificial Sequence
  Synthetic Oligonucleotide
aatttaatacgactcactatagggagatgtagtaggggctttcttctctgtcga

SEQ ID NO: 10 - DNA, 53 bp, Artificial Sequence
  Synthetic Oligonucleotide
aatttaatacgactcactatagggagagtagtaggggctttcttctctgtcga

SEQ ID NO: 11 - DNA, 55 bp, Artificial Sequence
  Synthetic Oligonucleotide
aatttaatacgactcactatagggagacatgtagtaggggctttcttctctgtcg

SEQ ID NO: 12 - DNA, 49 bp, Artificial Sequence
  Synthetic Oligonucleotide
aatttaatacgactcactatagggagagtaggggctttcttctctgtcg

SEQ ID NO: 13 - DNA, 47 bp, Artificial Sequence
  Synthetic Oligonucleotide
aatttaatacgactcactatagggagagcccaggtgcctgactacag

SEQ ID NO: 14 - DNA, 46 bp, Artificial Sequence
  Synthetic Oligonucleotide
aatttaatacgactcactatagggagagcccaggtgcctgactaca

SEQ ID NO: 15 - DNA, 46 bp, Artificial Sequence
  Synthetic Oligonucleotide
aatttaatacgactcactatagggagaggcccaggtgcctgactac

SEQ ID NO: 16 - DNA, 48 bp, Artificial Sequence
  Synthetic Oligonucleotide
aatttaatacgactcactatagggagagtttcgctttatctgaggacc

SEQ ID NO: 17 - DNA, 51 bp, Artificial Sequence
  Synthetic Oligonucleotide
aatttaatacgactcactatagggagagttagtttcgctttatctgaggac

SEQ ID NO: 18 - DNA, 53 bp, Artificial Sequence
  Synthetic Oligonucleotide
aatttaatacgactcactatagggagagttgttagtttcgctttatctgagga

SEQ ID NO: 19 - DNA, 49 bp, Artificial Sequence
  Synthetic Oligonucleotide
aatttaatacgactcactatagggagagactttccagaaactgttgtta

SEQ ID NO: 20 - DNA, 47 bp, Artificial Sequence
  Synthetic Oligonucleotide
aatttaatacgactcactatagggagaaactgaggtgggactttcca

SEQ ID NO: 21 - DNA, 51 bp, Artificial Sequence
  Synthetic Oligonucleotide
aatttaatacgactcactatagggagacttgaaactgaggtgggactttcc

SEQ ID NO: 22 - DNA, 54 bp, Artificial Sequence
  Synthetic Oligonucleotide
aatttaatacgactcactatagggagagtatttcccggtcttttggggaacttg

SEQ ID NO: 23 - DNA, 53 bp, Artificial Sequence
  Synthetic Oligonucleotide
aatttaatacgactcactatagggagagtatttcccggtcttttggggaactt

SEQ ID NO: 24 - DNA, 52 bp, Artificial Sequence
  Synthetic Oligonucleotide
aatttaatacgactcactatagggagagtatttcccggtcttttggggaact

SEQ ID NO: 25 - DNA, 23 bp, Artificial Sequence
  Synthetic Oligonucleotide
gtgccttcatcttttgagggctc

SEQ ID NO: 26 - DNA, 26 bp, Artificial Sequence
  Synthetic Oligonucleotide
ggagtgccttcatcttttgagggctc

SEQ ID NO: 27 - DNA, 22 bp, Artificial Sequence
  Synthetic Oligonucleotide
ggagtgccttcatcttttgagg

SEQ ID NO: 28 - DNA, 27 bp, Artificial Sequence
  Synthetic Oligonucleotide
gtcgaggagtgccttcatcttttgagg

SEQ ID NO: 29 - DNA, 26 bp, Artificial Sequence
  Synthetic Oligonucleotide
gtcgaggagtgccttcatcttttgag

SEQ ID NO: 30 - DNA, 28 bp, Artificial Sequence
  Synthetic Oligonucleotide
ctgtcgaggagtgccttcatcttttgag

SEQ ID NO: 31 - DNA, 27 bp, Artificial Sequence
  Synthetic Oligonucleotide
gtagtaggggctttcttctctgtcgag

SEQ ID NO: 32 - DNA, 24 bp, Artificial Sequence
  Synthetic Oligonucleotide
gtaggggctttcttctctgtcgag

SEQ ID NO: 33 - DNA, 27 bp, Artificial Sequence
  Synthetic Oligonucleotide
tgtagtaggggctttcttctctgtcga

SEQ ID NO: 34 - DNA, 26 bp, Artificial Sequence
  Synthetic Oligonucleotide
gtagtaggggctttcttctctgtcga

SEQ ID NO: 35 - DNA, 28 bp, Artificial Sequence
  Synthetic Oligonucleotide
catgtagtaggggctttcttctctgtcg

SEQ ID NO: 36 - DNA, 22 bp, Artificial Sequence
  Synthetic Oligonucleotide
gtaggggctttcttctctgtcg

SEQ ID NO: 37 - DNA, 20 bp, Artificial Sequence
  Synthetic Oligonucleotide
gcccaggtgcctgactacag

SEQ ID NO: 38 - DNA, 19 bp, Artificial Sequence
  Synthetic Oligonucleotide
gcccaggtgcctgactaca

SEQ ID NO: 39 - DNA, 19 bp, Artificial Sequence
  Synthetic Oligonucleotide
ggcccaggtgcctgactac

SEQ ID NO: 40 - DNA, 21 bp, Artificial Sequence
  Synthetic Oligonucleotide
gtttcgctttatctgaggacc

SEQ ID NO: 41 - DNA, 24 bp, Artificial Sequence
  Synthetic Oligonucleotide
gttagtttcgctttatctgaggac

SEQ ID NO: 42 - DNA, 26 bp, Artificial Sequence
  Synthetic Oligonucleotide
gttgttagtttcgctttatctgagga

SEQ ID NO: 43 - DNA, 22 bp, Artificial Sequence
  Synthetic Oligonucleotide
gactttccagaaactgttgtta

SEQ ID NO: 44 - DNA, 20 bp, Artificial Sequence
  Synthetic Oligonucleotide
aactgaggtgggactttcca

SEQ ID NO: 45 - DNA, 25 bp, Artificial Sequence
  Synthetic Oligonucleotide
acttgaaactgaggtgggactttcc

SEQ ID NO: 46 - DNA, 27 bp, Artificial Sequence
  Synthetic Oligonucleotide
gtatttcccggtcttttggggaacttg

SEQ ID NO: 47 - DNA, 26 bp, Artificial Sequence
  Synthetic Oligonucleotide
gtatttcccggtcttttggggaactt

SEQ ID NO: 48 - DNA, 25 bp, Artificial Sequence
  Synthetic Oligonucleotide
gtatttcccggtcttttggggaact

SEQ ID NO: 49 - DNA, 27 bp, Artificial Sequence
  Synthetic Oligonucleotide
  promoter (1)..(27)
aatttaatacgactcactatagggaga

SEQ ID NO: 50 - DNA, 18 bp, Artificial Sequence
  Synthetic Oligonucleotide
atgcccgatcagtccgtg

SEQ ID NO: 51 - DNA, 20 bp, Artificial Sequence
  Synthetic Oligonucleotide
tgatgcccgatcagtccgtg

SEQ ID NO: 52 - DNA, 18 bp, Artificial Sequence
  Synthetic Oligonucleotide
gatgcccgatcagtccgt

SEQ ID NO: 53 - DNA, 18 bp, Artificial Sequence
  Synthetic Oligonucleotide
tgatgcccgatcagtccg

SEQ ID NO: 54 - DNA, 20 bp, Artificial Sequence
  Synthetic Oligonucleotide
tgtgatgcccgatcagtccg

SEQ ID NO: 55 - DNA, 18 bp, Artificial Sequence
  Synthetic Oligonucleotide
gtgatgcccgatcagtcc

SEQ ID NO: 56 - DNA, 19 bp, Artificial Sequence
  Synthetic Oligonucleotide
tgggtcctacaaggcaaac

SEQ ID NO: 57 - DNA, 19 bp, Artificial Sequence
  Synthetic Oligonucleotide
agccacctacaatcagaca

SEQ ID NO: 58 - DNA, 33 bp, Artificial Sequence
  Synthetic Oligonucleotide
caaaagttacaaggaagtttaattaaagaataa

SEQ ID NO: 59 - DNA, 32 bp, Artificial Sequence
  Synthetic Oligonucleotide
caaaagttacaaggaagtttaattaaagaata

SEQ ID NO: 60 - DNA, 25 bp, Artificial Sequence
  Synthetic Oligonucleotide
ctcaaaagttacaaggaagtttaat

SEQ ID NO: 61 - DNA, 24 bp, Artificial Sequence
  Synthetic Oligonucleotide
ctcaaaagttacaaggaagtttaa

SEQ ID NO: 62 - DNA, 18 bp, Artificial Sequence
  Synthetic Oligonucleotide
gcttagcacgctagctac

SEQ ID NO: 63 - DNA, 18 bp, Artificial Sequence
  Synthetic Oligonucleotide
ggcttagcacgctagcta

SEQ ID NO: 64 - DNA, 19 bp, Artificial Sequence
  Synthetic Oligonucleotide
caccataaggcttagcacg

SEQ ID NO: 65 - DNA, 18 bp, Artificial Sequence
  Synthetic Oligonucleotide
caccataaggcttagcac

SEQ ID NO: 66 - DNA, 19 bp, Artificial Sequence
  Synthetic Oligonucleotide
acucccuacacagacucac

SEQ ID NO: 67 - DNA, 20 bp, Artificial Sequence
  Synthetic Oligonucleotide
cugaauaacacugggacagg

SEQ ID NO: 68 - DNA, 52 bp, Artificial Sequence
  Synthetic Oligonucleotide
uggcuuugcuggcauuuactttaaaaaaaaaaaaaaaaaaaaaaaaaaaaaa

SEQ ID NO: 69 - DNA, 58 bp, Artificial Sequence
  Synthetic Oligonucleotide
ugccccaauuuuggcuuugcuggcatttaaaaaaaaaaaaaaaaaaaaaaaaaaaaaa

SEQ ID NO: 70 - DNA, 57 bp, Artificial Sequence
  Synthetic Oligonucleotide
uccuuuugucugauuguagguggctttaaaaaaaaaaaaaaaaaaaaaaaaaaaaaa

SEQ ID NO: 71 - DNA, 56 bp, Artificial Sequence
  Synthetic Oligonucleotide
aguucccguagucuuuugagauctttaaaaaaaaaaaaaaaaaaaaaaaaaaaaaa

SEQ ID NO: 72 - DNA, 33 bp, Artificial Sequence
  Synthetic Oligonucleotide
tttaaaaaaaaaaaaaaaaaaaaaaaaaaaaaa

SEQ ID NO: 73 - DNA, 19 bp, Artificial Sequence
  Synthetic Oligonucleotide
uggcuuugcuggcauuuac

SEQ ID NO: 74 - DNA, 25 bp, Artificial Sequence
  Synthetic Oligonucleotide
ugccccaauuuuggcuuugcuggca

SEQ ID NO: 75 - DNA, 24 bp, Artificial Sequence
  Synthetic Oligonucleotide
uccuuuugucugauuguagguggc

SEQ ID NO: 76 - DNA, 23 bp, Artificial Sequence
  Synthetic Oligonucleotide
aguucccguagucuuuugagauc

SEQ ID NO: 77 - DNA, 52 bp, Artificial Sequence
  Synthetic Oligonucleotide
gggcuaggacuggggagcatttaaaaaaaaaaaaaaaaaaaaaaaaaaaaaa

SEQ ID NO: 78 - DNA, 54 bp, Artificial Sequence
  Synthetic Oligonucleotide
gaguguggaguucuuaccccutttaaaaaaaaaaaaaaaaaaaaaaaaaaaaaa

SEQ ID NO: 79 - DNA, 51 bp, Artificial Sequence
  Synthetic Oligonucleotide
cugguacuuuuccaugcctttaaaaaaaaaaaaaaaaaaaaaaaaaaaaaa

SEQ ID NO: 80 - DNA, 57 bp, Artificial Sequence
  Synthetic Oligonucleotide
guuuacuguagcuagcgugcuaagtttaaaaaaaaaaaaaaaaaaaaaaaaaaaaaa

SEQ ID NO: 81 - DNA, 19 bp, Artificial Sequence
  Synthetic Oligonucleotide
gggcuaggacuggggagca

SEQ ID NO: 82 - DNA, 21 bp, Artificial Sequence
  Synthetic Oligonucleotide
gaguguggaguucuuaccccu

SEQ ID NO: 83 - DNA, 18 bp, Artificial Sequence
  Synthetic Oligonucleotide
cugguacuuuuccaugcc

SEQ ID NO: 84 - DNA, 24 bp, Artificial Sequence
  Synthetic Oligonucleotide
guuuacuguagcuagcgugcuaag

SEQ ID NO: 85 - DNA, 8165 bp, Xenotropic MuLV-related virus VP62
  ref <308>: DATABASE ACCESSION NUMBER: GenBank/EF185282.1
  ref <309>: DATABASE ENTRY DATE: 2007-01-31
  ref <313>: RELEVANT RESIDUES IN SEQ ID NO: (1)..(8165)
agtcatccgatagactgagtcgcccgggtacccgtgttcccaataaagccttttgctgtttgcatccgaagcgtggcctcgctgttccttgggagggtctcctcagagtgattgactacccagctcgggggtctttcatttgggggctcgtccgggattcggagacccccgcccagggaccaccgacccaccgtcgggaggtaagccggccggcgatcgttttgtctttgtctctgtctttgtgcgtgtgtgtgtgtgccggcatctaatcctcgcgcctgcgtctgaatctgtactagttagctaactagatctgtatctggcggttccgcggaagaactgacgagttcgtattcccggccgcagccctgggagacgtcccagcggcctcgggggcccgttttgtggcccattctgtatcagttaacctacccgagtcggactttttggagtggctttgttgggggacgagagacagagacacttcccgcccccgtctgaatttttgctttcggttttacgccgaaaccgcgccgcgcgtctgatttgttttgttgttcttctgttcttcgttagttttcttctgtctttaagtgttctcgagatcatgggacagaccgtaactacccctctgagtctaaccttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccctctataaagtccaaacctcctaagccccaggttctccctgatagcggcggacctctcattgaccttctcacagaggatcccccgccgtacggagcacaaccttcctcctctgccagggagaacaatgaagaagaggcggccaccacctccgaggtttcccccccttctcccatggtgtctcgactgcggggaaggagagaccctcccgcagcggactccaccacctcccaggcattcccactccgcatggggggagatggccagcttcagtactggccgttttcctcctctgatttatataattggaaaaataataacccttccttttctgaagatccaggtaaattgacggccttgattgagtccgtcctcatcacccaccagcccacctgggacgactgtcagcagttgttggggaccctgctgaccggagaagaaaagcagcgggtgctcctagaggctagaaaggcagtccggggcaatgatggacgccccactcagttgcctaatgaagtcaatgctgcttttccccttgagcgccccgattgggattacaccactacagaaggtaggaaccacctagtcctctaccgccagttgctcttagcgggtctccaaaacgcgggcaggagccccaccaatttggccaaggtaaaagggataacccagggacctaatgagtctccctcagcctttttagagagactcaaggaggcctatcgcaggtacactccttatgaccctgaggacccagggcaagaaaccaatgtgtccatgtcattcatctggcagtctgccccggatatcggacgaaagttagagcggttagaagatttaaagagcaagaccttaggagacttagtgagggaagctgaaaagatctttaataagcgagaaaccccggaagaaagagaggaacgtatcaggagagaaatagaggaaaaagaagaacgccgtagggcagaggatgagcagagagagagagaaagggaccgcagaagacatagagagatgagcaagctcttggccactgtagttattggtcagagacaggatagacaggggggagagcggaggaggccccaacttgataaggaccaatgcgcctactgcaaagaaaagggacactgggctaaggactgcccaaagaagccacgagggccccgaggaccgaggccccagacctccctcctgaccttaggtgactagggaggtcagggtcaggagcccccccctgaacccaggataaccctcaaagtcggggggcaacccgtcaccttcctggtagatactggggcccaacactccgtgctgacccaaaatcctggacccctaagtgacaagtctgcctgggtccaaggggctactggaggaaagcggtatcgctggaccacggatcgcaaagtacatctggctaccggtaaggtcacccactctttcctccatgtaccagactgcccctatcctctgctaggaagagacttgctgactaaactaaaagcccaaatccacttcgagggatcaggagctcaggttgtgggaccgatgggacagcccctgcaagtgctgaccctaaacatagaagatgagtatcggctacatgagacctcaaaagagccagatgttcctctagggtccacatggctttctgattttccccaggcctgggcggaaaccgggggcatgggactggcagttcgccaagctcctctgatcatacctctgaaggcaacctctacccccgtgtccataaaacaataccccatgtcacaagaagccagactggggatcaagccccacatacagaggctgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaactgacctggaccagactcccacagggtttcaaaaacagtcccaccctgtttgatgaggcactgcacagagacctagcagatttccggatccagcacccagacttgatcctgctacagtacgtggatgacttactgctggccgccacttctgagcaagactgccaacgaggtactcgggccctattacaaaccctagggaacctcgggtatcgggcctcggccaagaaagcccaaatttgccagaaacaggtcaagtatctggggtatctcctaaaagagggacagagatggctgactgaggccagaaaagagactgtgatggggcagcccactccgaagacccctcgacaactaagggagttcctagggacggcaggcttctgtcgcctctggatccctgggtttgcagaaatggcagcccccttgtaccctcttaccaaaacggggactctgtttaattggggcccagaccagcaaaaggcctatcaagaaatcaaacaggctcttctaactgcccccgccctgggattgccagatttgactaagccctttgaactctttgtcgacgagaagcagggctacgccaaaggcgtcctaacgcaaaaactgggaccttggcgtcggcctgtggcctacctgtccaaaaagctagacccagtggcagctgggtggcccccttgcctacggatggtagcagccattgccgttctgacaaaggatgcaggcaagctaactatgggacagccgctagtcattctggccccccatgcggtagaagcactggtcaaacaaccccctgaccgttggctatccaatgcccgcatgacccactatcaggcaatgctcctggatacagaccgggttcagttcggaccggtggtggccctcaacccggccaccctgctccccctaccggaaaaggaagccccccatgactgcctcgagatcttggctgagacgcacggaaccagaccggacctcacggaccagcccatcccagacgctgattacacttggtacacagatggaagcagcttcctacaagaaggacaacggagagctggagcagcggtgactactgagaccgaggtaatctgggcgagggctctgccggctggaacatccgcccaacgagccgaactgatagcactcacccaagccttaaagatggcagaaggtaagaagctaaatgtttacactgatagccgctatgccttcgccacggcccatgtccatggagaaatatataggaggcgagggttgctgacctcagaaggcagagaaattaaaaacaagaacgagatcttggccttgctaaaagctctctttctgcccaaacgacttagtataattcactgtccaggacatcaaaaaggaaacagtgctgaggccagaggcaaccgtatggcagatcaagcagcccgagaggcagccatgaaggcagttctagaaacctctacactcctcatagaggactcaaccccgtatacgcctccccatttccattacaccgaaacagatctcaaaagactacgggaactgggagccacctacaatcagacaaaaggatattgggtcctacaaggcaaacctgtgatgcccgatcagtccgtgtttgaactgttagactccctacacagactcacccatctgagccctcaaaagatgaaggcactcctcgacagagaagaaagcccctactacatgttaaaccgggacagaactatccagtatgtgactgagacctgcaccgcctgtgcccaagtaaatgccagcaaagccaaaattggggcaggggtgcgagtacgcggacatcggccaggcacccattgggaagttgatttcacggaagtaaagccaggactgtatgggtacaagtacctcctagtgtttgtagacaccttctctggctgggtagaggcattcccgaccaagcgggaaactgccaaggtcgtgtccaaaaagctgttagaagacatttttccgagatttggaatgccgcaggtattgggatctgataacgggcctgccttcgcctcccaggtaagtcagtcagtggccgatttactggggatcgattggaagttacattgtgcttatagaccccagagttcaggacaggtagaaagaatgaatagaacaattaaggagactttgaccaaattaacgcttgcatctggcactagagactgggtactcctactccccttagccctctaccgagcccggaatactccgggcccccacggactgactccgtatgaaattctgtatggggcacccccgccccttgtcaattttcatgatcctgaaatgtcaaagttaactaatagtccctctctccaagctcacttacaggccctccaagcagtacaacaagaggtctggaagccgctggccgctgcttatcaggaccagctagatcagccagtgataccacaccccttccgtgtcggtgacgccgtgtgggtacgccggcaccagactaagaacttagaacctcgctggaaaggaccctacaccgtcctgctgacaacccccaccgctctcaaagtagacggcatctctgcgtggatacacgccgctcacgtaaaggcggcgacaactcctccggccggaacagcatggaaagtccagcgttctcaaaaccccttaaagataagattaacccgtggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatgggacctaatttcccttaagcgaggaaacactcctaagggtcagggcccctgttttgattcctcagtgggctccggtagcatccagggtgccacaccggggggtcgatgcaaccccctagtcctagaattcactgacgcgggtaaaagggccagctgggatgcccccaaaacatggggactaagactgtatcgatccactggggccgacccggtgaccctgttctctctgacccgccaggtcctcaatgtagggccccgcgtccccattgggcctaatcccgtgatcactgaacagctacccccctcccaacccgtgcagatcatgctccccaggcctcctcgtcctcctccttcaggcgcggcctctatggtgcctggggctcccccgccttctcaacaacctgggacgggagacaggctgctaaacctggtagaaggagcctaccaagccctcaacctcaccagtcccgacaaaacccaagagtgctggctgtgtctagtatcgggacccccctactacgaaggggtggccgtcctaggtacttactccaaccatacctctgccccggctaactgctccgtgacctcccaacacaagctgaccctgtccgaagtgaccgggcagggactctgcataggagcagttcccaaaacccatcaggccctgtgtaataccacccagaagacgagcgacgggtcctactatttggcctctcccgccgggaccatttgggcttgcagcaccgggctcactccctgtctatctactactgtgcttaacttaaccactgattactgtgtcctggttgaactctggccaaaggtaacctaccactcccctaattatgtttatggccagtttgaaaagaaaactaaatataaaagagagccggtgtcattaactctggccctgctgttgggaggacttactatgggcggcatagctgcaggagttggaacagggactacagccctagtggccaccaaacaattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcacgacaagggtggtttgagggactgtttaacaggtccccatggttcacgaccctgatatccaccattatgggccctctgatagtacttttattaatcctactcttcggaccctgtattctcaaccgcttggtccagtttgtaaaagacagaatttcggtagtgcaggccctggttctgacccaacagtatcaccaactcaaatcaatagatccagaagaagtggaatcacgtgaataaaagatttattcagtttccagaaagaggggggaatgaaagaccccaccataaggcttagcacgctagctacagtaacgccattttgcaagcatggaaaagtaccagagctgagttctcaaaagttacaaggaagtttaattaaagaataaggctgaataacactgggacaggggccaaacaggatatctgtagtcaggcacctgggccccggctcagggccaagaacagatggtcctcagataaagcgaaactaacaacagtttctggaaagtcccacctcagtttcaagttccccaaaagaccgggaaataccccaagccttatttaaactaaccaatcagctcgcttctcgcttctgtacccgcgctttttgctccccagtcctagccctataaaaaaggggtaagaactccacactcggcgcgccagtcatccgatagactgagtcgcccgggtacccgtgttcccaataaagcctt

SEQ ID NO: 86 - DNA, 18 bp, Artificial Sequence
  Synthetic Oligonucleotide
ctcaaaagatgaaggcac

SEQ ID NO: 87 - DNA, 19 bp, Artificial Sequence
  Synthetic Oligonucleotide
cctcaaaagatgaaggcac

SEQ ID NO: 88 - DNA, 23 bp, Artificial Sequence
  Synthetic Oligonucleotide
tcgacagagaagaaagcccctac

SEQ ID NO: 89 - DNA, 15 bp, Artificial Sequence
  Synthetic Oligonucleotide
atgcccgatcagtcc

SEQ ID NO: 90 - skipped

SEQ ID NO: 91 - DNA, 22 bp, Artificial Sequence
  Synthetic Oligonucleotide
caaaagttacaaggaagtttaa

SEQ ID NO: 92 - DNA, 18 bp, Artificial Sequence
  Synthetic Oligonucleotide
gtagtcaggcacctgggc

SEQ ID NO: 93 - DNA, 20 bp, Artificial Sequence
  Synthetic Oligonucleotide
gtcctcagataaagcgaaac

SEQ ID NO: 94 - DNA, 14 bp, Artificial Sequence
  Synthetic Oligonucleotide
tgccagcaaagcca

SEQ ID NO: 95 - DNA, 10 bp, Artificial Sequence
  Synthetic Oligonucleotide
cccccccccc

SEQ ID NO: 96 - DNA, 10 bp, Artificial Sequence
  Synthetic Oligonucleotide
gggggggggg

SEQ ID NO: 97 - DNA, 21 bp, Artificial Sequence
  Synthetic Oligonucleotide
accctgccagtccccctggaa

SEQ ID NO: 98 - DNA, 20 bp, Artificial Sequence
  Synthetic Oligonucleotide
accctgccagtccccctgga

SEQ ID NO: 99 - DNA, 18 bp, Artificial Sequence
  Synthetic Oligonucleotide
accctgccagtccccctg

SEQ ID NO: 100 - DNA, 24 bp, Artificial Sequence
  Synthetic Oligonucleotide
gggaatactggtaccctgccagtc

SEQ ID NO: 101 - DNA, 24 bp, Artificial Sequence
  Synthetic Oligonucleotide
gggaatactggtaccttgccagtc

SEQ ID NO: 102 - DNA, 22 bp, Artificial Sequence
  Synthetic Oligonucleotide
gaatactggtaccttgccagtc

SEQ ID NO: 103 - DNA, 17 bp, Artificial Sequence
  Synthetic Oligonucleotide
aagccccacatacagag

SEQ ID NO: 104 - DNA, 18 bp, Artificial Sequence
  Synthetic Oligonucleotide
caagccccacatacagag

SEQ ID NO: 105 - DNA, 23 bp, Artificial Sequence
  Synthetic Oligonucleotide
cgtgaataaaagattttattcag

SEQ ID NO: 106 - DNA, 24 bp, Artificial Sequence
  Synthetic Oligonucleotide
cgtgaataaaagattttattcagt

SEQ ID NO: 107 - DNA, 23 bp, Artificial Sequence
  Synthetic Oligonucleotide
cacgtgaataaaagattttattc

SEQ ID NO: 108 - DNA, 26 bp, Artificial Sequence
  Synthetic Oligonucleotide
gattttattcagtttccagaaagagg

SEQ ID NO: 109 - DNA, 23 bp, Artificial Sequence
  Synthetic Oligonucleotide
gattttattcagtttccagaaag

SEQ ID NO: 110 - DNA, 48 bp, Artificial Sequence
  Synthetic Oligonucleotide
  promoter (1)..(27)
aatttaatacgactcactatagggagatccacccgcttgttgacttct

SEQ ID NO: 111 - DNA, 48 bp, Artificial Sequence
  Synthetic Oligonucleotide
  promoter (1)..(27)
aatttaatacgactcactatagggagacacccgcttgttgacttctct

SEQ ID NO: 112 - DNA, 46 bp, Artificial Sequence
  Synthetic Oligonucleotide
  promoter (1)..(27)
aatttaatacgactcactatagggagatgtcttccacccgcttgtt

SEQ ID NO: 113 - DNA, 45 bp, Artificial Sequence
  Synthetic Oligonucleotide
  promoter (1)..(27)
aatttaatacgactcactatagggagatgtcttccacccgcttgt

SEQ ID NO: 114 - DNA, 46 bp, Artificial Sequence
  Synthetic Oligonucleotide
  promoter (1)..(27)
aatttaatacgactcactatagggagagttgggcacggtggggtgg

SEQ ID NO: 115 - DNA, 45 bp, Artificial Sequence
  Synthetic Oligonucleotide
  promoter (1)..(27)
aatttaatacgactcactatagggagagttgggcacggtggggtg

SEQ ID NO: 116 - DNA, 51 bp, Artificial Sequence
  Synthetic Oligonucleotide
  promoter (1)..(27)
aatttaatacgactcactatagggagaatgccttgcaaaatggcgttactg

SEQ ID NO: 117 - DNA, 50 bp, Artificial Sequence
  Synthetic Oligonucleotide
  promoter (1)..(27)
aatttaatacgactcactatagggagaatgccttgcaaaatggcgttact

SEQ ID NO: 118 - DNA, 49 bp, Artificial Sequence
  Synthetic Oligonucleotide
  promoter (1)..(27)
aatttaatacgactcactatagggagaatgccttgcaaaatggcgttac

SEQ ID NO: 119 - DNA, 44 bp, Artificial Sequence
  Synthetic Oligonucleotide
  promoter (1)..(27)
aatttaatacgactcactatagggagattttccatgccttgcaa

SEQ ID NO: 120 - DNA, 43 bp, Artificial Sequence
  Synthetic Oligonucleotide
  promoter (1)..(27)
aatttaatacgactcactatagggagagaactcagctctggta

SEQ ID NO: 121 - DNA, 45 bp, Artificial Sequence
  Synthetic Oligonucleotide
  promoter (1)..(27)
aatttaatacgactcactatagggagatgagaactcagctctggt

SEQ ID NO: 122 - DNA, 21 bp, Artificial Sequence
  Synthetic Oligonucleotide
tccacccgcttgttgacttct

SEQ ID NO: 123 - DNA, 21 bp, Artificial Sequence
  Synthetic Oligonucleotide
cacccgcttgttgacttctct

SEQ ID NO: 124 - DNA, 19 bp, Artificial Sequence
  Synthetic Oligonucleotide
tgtcttccacccgcttgtt

SEQ ID NO: 125 - DNA, 18 bp, Artificial Sequence
  Synthetic Oligonucleotide
tgtcttccacccgcttgt

SEQ ID NO: 126 - DNA, 19 bp, Artificial Sequence
  Synthetic Oligonucleotide
gttgggcacggtggggtgg

SEQ ID NO: 127 - DNA, 18 bp, Artificial Sequence
  Synthetic Oligonucleotide
gttgggcacggtggggtg

SEQ ID NO: 128 - DNA, 24 bp, Artificial Sequence
  Synthetic Oligonucleotide
atgccttgcaaaatggcgttactg

SEQ ID NO: 129 - DNA, 23 bp, Artificial Sequence
  Synthetic Oligonucleotide
atgccttgcaaaatggcgttact

SEQ ID NO: 130 - DNA, 22 bp, Artificial Sequence
  Synthetic Oligonucleotide
atgccttgcaaaatggcgttac

SEQ ID NO: 131 - DNA, 17 bp, Artificial Sequence
  Synthetic Oligonucleotide
ttttccatgccttgcaa

SEQ ID NO: 132 - DNA, 16 bp, Artificial Sequence
  Synthetic Oligonucleotide
gaactcagctctggta

SEQ ID NO: 133 - DNA, 18 bp, Artificial Sequence
  Synthetic Oligonucleotide
tgagaactcagctctggt

SEQ ID NO: 134 - DNA, 59 bp, Artificial Sequence
  Synthetic Oligonucleotide
ugugacaugggguauuguuuuauggatttaaaaaaaaaaaaaaaaaaaaaaaaaaaaaa

SEQ ID NO: 135 - DNA, 59 bp, Artificial Sequence
  Synthetic Oligonucleotide
uggcuucuugugacaugggguauuguuttaaaaaaaaaaaaaaaaaaaaaaaaaaaaaa

SEQ ID NO: 136 - DNA, 59 bp, Artificial Sequence
  Synthetic Oligonucleotide
cucaaaggcgaagagaggcugacuggtttaaaaaaaaaaaaaaaaaaaaaaaaaaaaaa

SEQ ID NO: 137 - DNA, 53 bp, Artificial Sequence
  Synthetic Oligonucleotide
aucuguucuuggcccugagctttaaaaaaaaaaaaaaaaaaaaaaaaaaaaaa

SEQ ID NO: 138 - DNA, 56 bp, Artificial Sequence
  Synthetic Oligonucleotide
accaucuguucuuggcccugagctttaaaaaaaaaaaaaaaaaaaaaaaaaaaaaa

SEQ ID NO: 139 - DNA, 56 bp, Artificial Sequence
  Synthetic Oligonucleotide
acuaucuguucuuggcccugagctttaaaaaaaaaaaaaaaaaaaaaaaaaaaaaa

SEQ ID NO: 140 - DNA, 55 bp, Artificial Sequence
  Synthetic Oligonucleotide
uacagaagcgagaagcgagcugtttaaaaaaaaaaaaaaaaaaaaaaaaaaaaaa

SEQ ID NO: 141 - RNA, 26 nt, Artificial Sequence
  Synthetic Oligonucleotide
ugugacaugggguauuguuuuaugga

SEQ ID NO: 142 - RNA, 27 nt, Artificial Sequence
  Synthetic Oligonucleotide
uggcuucuugugacaugggguauuguu

SEQ ID NO: 143 - RNA, 26 nt, Artificial Sequence
  Synthetic Oligonucleotide
cucaaaggcgaagagaggcugacugg

SEQ ID NO: 144 - RNA, 20 nt, Artificial Sequence
  Synthetic Oligonucleotide
aucuguucuuggcccugagc

SEQ ID NO: 145 - RNA, 23 nt, Artificial Sequence
  Synthetic Oligonucleotide
accaucuguucuuggcccugagc

SEQ ID NO: 146 - RNA, 23 nt, Artificial Sequence
  Synthetic Oligonucleotide
acuaucuguucuuggcccugagc

SEQ ID NO: 147 - RNA, 22 nt, Artificial Sequence
  Synthetic Oligonucleotide
uacagaagcgagaagcgagcug

SEQ ID NO: 148 - RNA, 20 nt, Artificial Sequence
  Synthetic Oligonucleotide
cuacccguuaagaaaccagg

SEQ ID NO: 149 - RNA, 26 nt, Artificial Sequence
  Synthetic Oligonucleotide
cuacccguuaagaaaccagggacuaa

SEQ ID NO: 150 - RNA, 21 nt, Artificial Sequence
  Synthetic Oligonucleotide
cguuaagaaaccagggacuaa

SEQ ID NO: 151 - DNA, 18 bp, Artificial Sequence
  Synthetic Oligonucleotide
cagaaagaggggggaatg

SEQ ID NO: 152 - RNA, 18 nt, Artificial Sequence
  Synthetic Oligonucleotide
  misc_feature (12)..(12): n is inosine
cagaaagagggnggaaug

SEQ ID NO: 153 - RNA, 18 nt, Artificial Sequence
  Synthetic Oligonucleotide
cauuccccccucuuucug

SEQ ID NO: 154 - RNA, 15 nt, Artificial Sequence
  Synthetic Oligonucleotide
ggaaugaaagacccc

SEQ ID NO: 155 - RNA, 20 nt, Artificial Sequence
  Synthetic Oligonucleotide
gaccccaccauaaggcuuag

SEQ ID NO: 156 - RNA, 20 nt, Artificial Sequence
  Synthetic Oligonucleotide
gaccccuucauaaggcuuag

SEQ ID NO: 157 - DNA, 19 bp, Artificial Sequence
  Synthetic Oligonucleotide
cacccgcttgttgacttct

SEQ ID NO: 158 - DNA, 21 bp, Artificial Sequence
  Synthetic Oligonucleotide
cgtgaataaaagattttattc

SEQ ID NO: 159 - DNA, 15 bp, Artificial Sequence
  Synthetic Oligonucleotide
gaactcagctctggt